(19) United States
(12) Patent Application Publication
(10) Pub. No.: US 2024/0149245 A1
(43) Pub. Date: May 9, 2024

(54) ULTRASMALL NANOPARTICLES AND METHODS OF MAKING, USING AND ANALYZING SAME

(71) Applicant: CORNELL UNIVERSITY, Ithaca, NY (US)

(72) Inventors: Melik Z. Turker, Princeton, NJ (US); Thomas C. Gardinier, II, Raritan, NJ (US); Joshua A. Hinckley, Framingham, MA (US); Ulrich B. Wiesner, Ithaca, NY (US)

(21) Appl. No.: 17/773,267

(22) PCT Filed: Nov. 4, 2020
(86) PCT No.: PCT/US2020/058949

§ 371 Date: Apr. 29, 2022

Related U.S. Application Data
(60) Provisional application No. 62/930,539, filed on Nov. 4, 2019.

(51) Int. Cl. B01J 20/286; A61K 49/00; B01J 20/08; B01J 20/10; B01J 20/16; B01J 20/22; B01J 20/26; B01J 20/30; B01J 20/32; B82Y 30/00; B82Y 40/00; G01N 30/86

(52) U.S. Cl. CPC B01J 20/286; A61K 49/0093; B01J 20/08; B01J 20/103; B01J 20/16; B01J 20/22; B01J 20/262; B01J 20/3085; B01J 20/3204; B01J 20/3217; B01J 20/3272; B01J 20/3293; G01N 30/8624; B82Y 30/00; B82Y 40/00

(57) ABSTRACT

The present disclosure provides methods of analyzing and/or purifying inorganic nanoparticles that may be functionalized with one or more dye group. Analyzing and/or purifying the inorganic nanoparticles includes utilizing liquid chromatography, such as, for example, high performance liquid chromatography (HPLC). Methods of the present disclosure may be used to determine the location of one or more dye groups on and/or in the inorganic nanoparticles. The present disclosure also provides methods of making inorganic nanoparticles and compositions of inorganic nanoparticles.

### CROSS REFERENCE TO RELATED APPLICATION

This application claims priority to U.S. provisional application No. 62/930,539 filed Nov. 4, 2019, the disclosure of which is incorporated herein by reference.

### STATEMENT REGARDING FEDERALLY SPONSORED RESEARCH OR DEVELOPMENT

This invention was made with government support under grant numbers CA199081 and GM122575 awarded by the National Institutes of Health. The government has certain rights in the invention.

### BACKGROUND OF THE DISCLOSURE

Silica nanoparticles (SNPs) have attracted interest for potential therapeutic/diagnostic applications due to their large surface-area, inertness and high bio-compatibility. However, most SNPs are >10 nm in size.

Nanoparticle synthesis is ubiquitous in a host of research fields from energy to healthcare and provides access to a diverse array of materials such as quantum dots or polymer, metal, and oxide nanoparticles. Key characteristics of successful nanoparticle preparation methods are the batch-to-batch reproducibility and control over properties such as size, brightness, and surface chemistry. In the last five to ten years, increasing interest has focused on the synthesis of ultrasmall (diameter <10 nm) nanoparticles. In addition to unique properties emerging at this scale, their small size enables use of high performance liquid chromatography (HPLC) to quantitatively analyze particle surface chemical properties. While HPLC is ubiquitous in fields with precisely defined molecular materials such as small molecules, macromolecular structures like dendrimers, and proteins, the successful application of HPLC to inorganic core-organic shell (core-shell) nanoparticles is a recent development. As a result of the well-established versatility of HPLC for synthesis product quality control, this adds a novel and intriguing dimension to the analysis of nanoparticles, e.g., in order to further tune their surface chemical properties for biological applications. Within a single synthesis batch, HPLC allows mapping of variations in the surface chemistry of nanoparticles onto different peaks in the chromatograms. Such quantitative assessments of the degree of heterogeneity in particle surface chemical properties are in stark contrast to the averaged particle surface properties as typically revealed, e.g. via zeta potential or spectroscopic measurements. Furthermore, in combination with other analytical techniques such as gel-permeation chromatography (GPC), HPLC enables multidimensional correlation analyses. In the case of coupled GPC-HPLC runs, e.g., this allows to map surface chemical heterogeneities onto particle size dispersity. This in turn opens the door to answering questions of how particle batch heterogeneities modulate biological response, hitherto a largely unexplored area because of the lack of appropriate quantitative characterization techniques.

A system of particular interest for in-depth study with this type of analytical technique are Cornell prime dots (C′ dots), a class of ultrasmall (diameter <10 nm) fluorescent core-shell silica nanoparticles currently in multiple clinical trials to test for both diagnostic and therapeutic clinical potential [for targeted PET and/or optical detection of metastatic melanoma (NCT01266096, NCT03465618) and malignant brain tumors (NCT02106598)]. C′ dots are composed of a fluorescent dye covalently encapsulated within a silica core grown via sol-gel chemistry and covalently coated with a brush like poly(ethylene glycol)-(PEG-)silane shell. The encapsulated fluorescent dye can be varied based on application, but typically dyes such as Cy5 and Cy5.5 are used for their near infrared (NIR) absorption and emission profiles advantageous for biological applications. The underlying chemical structure of these dyes is extremely hydrophobic. Therefore, often sulfonate groups are introduced in the dye periphery in order to increase their hydrophilicity and facilitate their use in aqueous media.

In early iterations of the water-based C′ dot synthesis, typically sulfonated analogues of Cy5 and Cy5.5 dyes were conjugated to a silane moiety via a maleimide-thiol coupling reaction. The resulting dye-silane conjugates were then covalently encapsulated into the silica matrix via the sol-gel process and subsequently PEGylated, providing enhanced photophysical properties along with significantly increased hydrophilicity of the resulting core-shell dots relative to the free dye in water. Recent work employing HPLC to elucidate the surface chemistry of such C′ dots revealed, however, that use of negatively charged sulfonated Cy5 was the cause of significant surface chemical heterogeneity. HPLC chromatograms of such particles exhibited multiple peaks (see FIG. 2a below). The first peak at the shortest elution time corresponded to C′ dots with the desired purely PEGylated nanoparticle surface with Cy5 dye either fully encapsulated or completely absent. Subsequent peaks could be assigned to one, two, or three Cy5 dyes on the silica nanoparticle surface, respectively, leading to hydrophobic patches between the PEG chains, which in turn were responsible for the observed shifts to longer elution times in the HPLC chromatograms. These results were corroborated using additional techniques such as after-pulse corrected fluorescence correlation spectroscopy (FCS), single particle photobleaching, and molecular dynamics simulations. Dye charge was found to play a key role in the successful encapsulation of a specific dye into the silica matrix, rather than its covalent attachment onto the silica nanoparticle core surface.

There is an ongoing and unmet need for methods to produce inorganic nanoparticles with desirable features.

### SUMMARY OF THE DISCLOSURE

The present disclosure provides methods of analyzing and/or purifying inorganic nanoparticles (e.g., core or core-shell nanoparticles). The inorganic nanoparticles are also referred to herein as ultrasmall nanoparticles. The present disclosure also provides methods of making inorganic nanoparticles and compositions comprising inorganic nanoparticles.

In various examples, the present disclosure provides:
(1) The application of high performance liquid chromatography (HPLC) methods, which enabled the discovery of hitherto unknown surface-chemical heterogeneities in inorganic nanoparticles (e.g., fluorescent core-shell silica nanoparticles).
(2) The identification of design criteria to overcome the intrinsic nanoparticle heterogeneities observed.
(3) The discovery that fluorescent dye charge is a crucial parameter in the control of surface chemistry heterogeneity.
(4) The use of positively charged dyes, such as ATTO647N, MB2, all cyanine group dyes (e.g., Cy5 Cy5.5, Cy7, and the like) in some embodiments to synthesize C′ dots with highly homogeneous surface-chemical properties, displaying only a single peak in the HPLC analysis.
(5) These homogeneous C′ dots exhibit profoundly higher stability against chemical degradation.
(6) These homogeneous C′ dots exhibit profoundly higher photostability when compared to previously reported materials.

In certain examples, the present disclosure provides:
(1) Creation of an ultrasmall (sub-10 nm) nanoparticle with a completely homogeneous surface chemistry.
(2) Reduces dye sensitivity to solvent environment, normally hydrophobic dyes like ATTO647N cannot be reliably used in water-based applications.
(3) Uses for clinically relevant diagnostic imaging.
(4) Uses for improvement of bio-distribution of nanoparticle diagnostics and therapies
(5) Uses for other nanoparticles in the process of clinical translation.
(6) Useful to analyze surface chemistry engineering for PEGylated materials which was until now not straight forward.

In an aspect, the present disclosure provides the analysis and/or purification of inorganic nanoparticles via liquid chromatography. The synthesis, analysis, and/or purification may be carried out using HPLC and/or GPC. Also provided are synthetic pathways to make dyes fully encapsulated in nanoparticles.

Inorganic nanoparticles comprising various dye groups may be suitable for analysis and/or purification. The dye groups may be located in various locations on and/or in (encapsulated (fully encapsulated) by or partially encapsulated (fully encapsulated) by or encapsulated in or partially encapsulated in) an inorganic nanoparticle. The dye groups may be disposed or partially disposed on the surface of an inorganic nanoparticle, encapsulated (fully encapsulated) or partially encapsulated in/by the inorganic nanoparticle, or a combination thereof. A dye group disposed or partially disposed on the surface of an inorganic nanoparticle may refer to the dye group being part of a PEG group disposed or partially disposed on the surface of the inorganic nanoparticle.

Inorganic nanoparticles may be analyzed by high performance liquid chromatography (HPLC). HPLC may be used to determine the location of one or more dye group on and/or in (e.g., encapsulated by) an inorganic nanoparticle. Such a method may comprise subjecting a plurality of inorganic nanoparticles to HPLC analysis.

A composition comprising a plurality of inorganic nanoparticles may be purified using liquid chromatography. In an example, the liquid chromatography is GPC or preparative scale HPLC (e.g., preparative-scale RP-HPLC).

Methods of purification and/or analysis may generate eluent containing a purified, an analyzed, and/or a selected portion of inorganic nanoparticles. The purified, analyzed, and/or selected portion of inorganic nanoparticles may be referred to as fractions. The fractions may be combined to generate various compositions comprising desirable combinations of inorganic nanoparticles. For example, a fraction containing a plurality of inorganic nanoparticles, where individual inorganic nanoparticles encapsulate one or more anionic dye groups, may be combined with a fraction containing a plurality of inorganic nanoparticles, where individual inorganic nanoparticles have one anionic dye group disposed or partially disposed on the exterior surface of the individual inorganic nanoparticles.

In an aspect, the present disclosure provides a method of making inorganic nanoparticles (e.g., ultrasmall nanoparticles). The methods are based on use of aqueous reaction medium (e.g. water). The nanoparticles can be surface functionalized with polyethylene glycol groups (e.g., PEGylated) and/or various dye groups. A dye group disposed or partially disposed on the surface of an inorganic nanoparticle may refer to the dye group being part of a PEG group disposed or partially disposed on the surface of the inorganic nanoparticle.

In an aspect, the present disclosure provides compositions comprising inorganic nanoparticles of the present disclosure. The compositions can comprise one or more types (e.g., having different average size and/or one or more different compositional feature).

In an aspect, the present disclosure provides uses of the inorganic nanoparticles and compositions of the present disclosure. For example, inorganic nanoparticles or a composition comprising the inorganic nanoparticles are used in delivery and/or imaging methods.

This disclosure provides a method for imaging biological material such as cells, extracellular components, or tissues comprising contacting the biological material with inorganic nanoparticles comprising one or more positively charged dyes, or compositions comprising the nanoparticles; directing excitation electromagnetic (e/m) radiation, such as light, on to the tissues or cells thereby exciting the positively charged dye molecules; detecting e/m radiation emitted by the excited positively charged dye molecules; and capturing and processing the detected e/m radiation to provide one or more images of the biological material. One or more of these steps can be carried out in vitro or in vivo. For example, the cells or tissues can be present in an individual or can be present in culture. Exposure of cells or tissues to e/m radiation can be effected in vitro (e.g., under culture conditions) or can be effected in vivo. For directing e/m radiation at cells, extracellular materials, tissues, organs and the like within an individual or any portion of an individual's body that are not easily accessible, fiber optical instruments can be used.

### BRIEF DESCRIPTION OF THE FIGURES

For a fuller understanding of the nature and objects of the disclosure, reference should be made to the following detailed description taken in conjunction with the accompanying figures.

FIG. 1 shows structures of sulfonated and unsulfonate Cy5 and Cy5.5 maleimide derivatives. The sulfonated Cy5 has a net charge of −1 in aqueous solution, while the unsulfonated derivatives of both Cy5-maleimide and Cy5.5-maleimide have a net charge of +1 in aqueous solution. The sulfonated form of Cy5.5-maleimide has a net charge of −3 to counteract the significant hydrophobicity of this dye structure.

FIG. 2 shows (a) HPLC chromatograms of PEG-sulfo-Cy5-C′ dots and PEG-Cy5(+)-C′ dots with schematic representation of the type of nanoparticles that elute in each peak, and highlighting that the PEG-Cy5(+)-C′ dot peak overlaps completely with the peak corresponding to purely PEGylated particles in the PEG-sulfo-Cy5-C′ dot sample. (b) HPLC chromatograms of PEG-sulfo-Cy5.5-C′ dots and PEG-Cy5.5(+)-C′ dots, again highlighting the complete overlap of the PEG-Cy5.5-C′ dots with the purely PEGylated particles in the PEG-sulfo-Cy5.5-C′ dot sample. (c) HPLC chromatograms of PEG-sulfo-Cy5-C′ dots and PEG-sulfo-Cy5.5-C′ dots highlighting the substantially more hydrophobic behavior of the latter. (d) HPLC chromatograms of PEG-Cy5(+)-C′ dots and PEG-Cy5.5(+)-C′ dots.

FIG. 3 shows (a-c) GPC (left row), HPLC (middle row), and FCS (right row) of PEG-Cy5(+)-C′ dots made with a starting ammonia concentration of 6 mM. (d-f) GPC, HPLC, and FCS of PEG-Cy5(+)-C′ dots made with a starting ammonia concentration of 5 mM. (g-i) GPC, HPLC, and FCS of PEG-Cy5(+)-C′ dots made with a starting ammonia concentration of 2 mM. (j-l) GPC, HPLC, and FCS of PEG-Cy5(+)-C′ dots made with a starting ammonia concentration of 1 mM.

FIG. 4 shows a schematic of silica nanoparticle growth via the aggregation of primary silica clusters around positively charged dyes. The halo around the silica clusters symbolizes the net negative cluster charge increasing with higher pH conditions.

FIG. 5 shows (a-c) GPC (left row), HPLC (middle row), and FCS (right row) of PEG-Cy5.5(+)-C′ dots made with a starting ammonia concentration of 5 mM. (d-f) GPC, HPLC, and FCS of PEG-Cy5.5(+)-C′ dots made with a starting ammonia concentration of 2 mM. (g-i) GPC, HPLC, and FCS of PEG-Cy5.5(+)-C′ dots made with a starting ammonia concentration of 1.5 mM. (j-l) GPC, HPLC, and FCS of PEG-Cy5.5(+)-C′ dots made with a starting ammonia concentration of 1 mM.

FIG. 6 shows (a) an HPLC chromatogram of PEG-Cy5.5-C′ dots synthesized at a starting ammonia concentration of 1 mM. (b) GPC of PEG-Cy5.5-C′ dots synthesized at a starting ammonia concentration of 1 mM, the shaded regions highlight the area under the curve that was fractionated and combined for GPC-HPLC of the sample in c-e. (c-e) HPLC chromatograms of GPC fractionated PEG-Cy5.5-C′ dots (c) are the largest nanoparticles, (d) are average sized nanoparticles, and (e) are the smallest nanoparticles from the GPC fractionation.

FIG. 7 shows (a) cell death experiments on MDA-MB-468 cells using iron(III) nitrate in both complete media (triangle) and in amino acid (AA) deprived media (square). (b) Cell death experiments on MDA-MB-468 cells in AA deprived media comparing the efficacy of PEG-Cy5(+)-C′ dots and PEG-sulfoCy5-C′ dots in the presence of a non-toxic amount of iron (1 μM).

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

The present disclosure provides methods of analyzing and/or purifying inorganic nanoparticles (e.g., core or core-shell nanoparticles). The inorganic nanoparticles are also referred to herein as ultrasmall nanoparticles. The present disclosure also provides methods of making inorganic nanoparticles and compositions comprising inorganic nanoparticles.

All ranges provided herein include all values that fall within the ranges to the tenth decimal place, unless indicated otherwise.

In various examples, the present disclosure provides:
(1) The application of high performance liquid chromatography (HPLC) methods, which enabled the discovery of hitherto unknown surface-chemical heterogeneities in inorganic nanoparticles (e.g., fluorescent core-shell silica nanoparticles).
(2) The identification of design criteria to overcome the intrinsic nanoparticle heterogeneities observed.
(3) The discovery that fluorescent dye charge is a crucial parameter in the control of surface chemistry heterogeneity.
(4) The use of positively charged dyes, such as ATTO647N and MB2 in some embodiments to synthesize C′ dots with highly homogeneous surface-chemical properties, displaying only a single peak in the HPLC analysis.
(5) These homogeneous C′ dots exhibit profoundly higher stability against chemical degradation.
(6) These homogeneous C′ dots exhibit profoundly higher photostability when compared to previously reported materials.

In certain examples, the present disclosure provides:
(1) Creation of an ultrasmall (sub-10 nm) nanoparticle with a completely homogeneous surface chemistry.
(2) Reduces dye sensitivity to solvent environment, normally hydrophobic dyes like ATTO647N cannot be reliably used in water-based applications.
(3) Uses for clinically relevant diagnostic imaging.
(4) Uses for improvement of bio-distribution of nanoparticle diagnostics and therapies
(5) Uses for other nanoparticles in the process of clinical translation.
(6) Useful to analyze surface chemistry engineering for PEGylated materials which was until now not straight forward.

In an aspect, the present disclosure provides the analysis and/or purification of inorganic nanoparticles via liquid chromatography. The analysis and/or purification may be carried out using HPLC and/or GPC.

Inorganic nanoparticles comprising various dye groups may be suitable for analysis and/or purification. The dye groups may be located in various locations on and/or in (encapsulated by or partially encapsulated by) an inorganic nanoparticle. The dye groups may be disposed or partially disposed on the surface of an inorganic nanoparticle, encapsulated or partially encapsulated by the inorganic nanoparticle, or a combination thereof. A dye group disposed or partially disposed on the surface of an inorganic nanoparticle may refer to the dye group being part of a PEG group disposed or partially disposed on the surface of the inorganic nanoparticle.

Inorganic nanoparticles may be analyzed by high performance liquid chromatography (HPLC). HPLC may be used to determine the location of one or more dye group on and/or in (e.g., encapsulated by) an inorganic nanoparticle. Such a method may comprise subjecting a plurality of inorganic nanoparticles to HPLC analysis.

A method of analyzing inorganic nanoparticles may comprise: (i) depositing an inorganic nanoparticle in an HPLC column comprising an input in fluid communication with a stationary phase in fluid communication with an output in fluid communication with a detector; (ii) passing a mobile phase through the HPLC column, such that the inorganic nanoparticle elutes from the column and enters the detector, such that the detector generates a signal, wherein the signal indicates the location of the one or more dye group on and/or in the nanoparticle and/or core-shell nanoparticle; and (iii) analyzing the signal to determine the location of the one or more dye group on and/or in the inorganic nanoparticle. The signal comprises a retention time that correlates to the location of one or more dye group on and/or in (e.g., encapsulated by or partially encapsulated by) an inorganic nanoparticle. A peak at a specific retention time may also correlate to the number of dye groups disposed and/or partially disposed on the exterior surface of an inorganic particle or whether dye groups are in (e.g., encapsulated by or partially encapsulated by) an inorganic nanoparticle. Methods of HPLC analysis of the present disclosure may be reproducible.

In an example, whenever eluent comprising inorganic nanoparticles passes through a detector, the detector generates a signal with an intensity greater than baseline. The relative time at which a signal occurs following the injection of a sample comprising a plurality of inorganic nanoparticles in the column determines the elution time of a portion of the plurality of inorganic nanoparticles. The elution time correlates to a portion of inorganic nanoparticles eluted from the column, with more hydrophobic particles being eluted at later times. Without intending to being bound by any particular theory, it is expected that an increasing number of hydrophobic dye group disposed on the surface of an inorganic nanoparticle increases the inorganic nanoparticle's elution time. As an illustrative example, a inorganic nanoparticle that has two hydrophobic dye groups disposed or partially disposed on the surface elutes later than an inorganic nanoparticle with only one dye disposed or partially disposed on the surface.

Various detectors are suitable for use in a method of analyzing an inorganic nanoparticle via HPLC. Examples of suitable detectors include, but are not limited to, a UV detector (e.g., a tunable UV detector), an evaporative light scattering detector, a charged aerosol detector, a fluorescence-based detector (e.g., a fluorimeter), a photodiode array detector, and the like, and combinations thereof.

Various HPLC columns are suitable for a method of analyzing an inorganic nanoparticle via HPLC. An HPLC column may be a reverse-phase HPLC column (RP-HPLC column). An RP-HPLC column may comprise a C4 stationary phase to a C8 stationary phase or other suitable moderately hydrophilic stationary phases (e.g., C4, C5, C6, C7, or C8 stationary phase). An RP-HPLC column may have various lengths. For example, a suitable RP-HPLC column is 100 to 300 mm long, including every integer mm value and range therebetween (e.g., 150-250 mm in length, such as, for example, 150 mm in length). An RP-HPLC column may have various pore sizes. For example, a suitable RP-HPLC column has a pore size of 200 to 400, including every integer A value and range therebetween (e.g., 250 to 350, such as, for example, 300 Å). An RP-HPLC column may have various particle sizes. For example, a suitable RP-HPLC column has a particle size of 2 to 6 m, including every 0.1 m value and range therebetween (e.g., 3.5 to 5 m). An RP-HPLC column may have various internal diameters. For example, an RP-HPLC may have an internal diameter of 4.6 mm. A mobile phase may be passed through an RP-HPLC column at various rates. For example, a mobile phase is passed through the column at a flow rate of 0.1 to 2.0 mL/min, including every 0.1 mL/min value and range therebetween (e.g., 0.5 to 1 mL/min). An RP-HPLC column may be maintained at various temperatures. For example, a suitable RP-HPLC column is maintained at 15 to 30° C., including every 0.1° C. value and range therebetween (e.g., 18 to 25° C.). In an example, the RP-HPLC column is not a C18 RP-HPLC column.

Various mobile phases are suitable for a method of analyzing an inorganic nanoparticle via HPLC. A mobile phase is an aqueous mobile phase, such as, for example, a water and acetonitrile mixture or a water and isopropanol and/or methanol mixture. A mobile phase may further comprise an acid, such as, for example, trifluoroacetic acid (TFA) or formic acid at a concentration of 0.01 to 1% by volume. Other suitable mobile phases are known in the art.

The mobile phase may be passed through the column in a step-like gradient. For example, a mobile phase comprising a polar portion and nonpolar portion, where polar portion exceeds the nonpolar portion (e.g., 90:10 water:acetonitrile) may be passed through an HPLC column at a flow rate of, for example, 1 mL/min. These conditions may be maintained for a period of time (e.g., 20 minutes) to allow equilibration of an analyte (e.g., an inorganic nanoparticle) with the stationary phase. After the period of time (e.g., 20 minutes) the flow rate may be decreased (e.g., to 0.5 mL/min) and the HPLC column may be allowed to equilibrate. The mobile phase composition may then be changed such that the nonpolar portion slightly exceeds the polar portion (e.g., 45:55 water:acetonitrile) in a step-like fashion and the baseline may be allowed to equilibrate again. Finally, a composition gradient of where the nonpolar portion is further increased may be used (e.g., 45:55 to 5:95 water:acetonitrile) for a period of time (e.g., 20 minutes), during which time the analyte (e.g., a selected portion of inorganic nanoparticles) elutes from the column.

A composition comprising a plurality of inorganic nanoparticles may be purified using liquid chromatography. In an example, the liquid chromatography is GPC or preparative scale HPLC (e.g., preparative-scale RP-HPLC).

Inorganic nanoparticles may be purified using gel permeation chromatography (GPC). GPC may be used to purify inorganic nanoparticles and/or determine separate batches of inorganic nanoparticles based on size.

A method of purifying inorganic nanoparticles may comprise: (i) depositing the plurality of inorganic nanoparticles in a chromatography column comprising an input in fluid communication with a stationary phase in fluid communication with an output in fluid communication with a detector; (ii) passing a mobile phase through the chromatography column, such that the plurality of inorganic nanoparticles elutes from the column; and (iii) collecting an eluent comprising the selected portion of the plurality inorganic nanoparticles.

The chromatography column may be a GPC column having a porous gel stationary phase. Other suitable stationary phases are known in the art. In an example, the mobile phase may be an aqueous mobile phase, such as, for example water, an aqueous solution of NaCl (e.g., a 0.9 wt % NaCl aqueous solution). Other suitable mobile phases are known in the art.

Inorganic nanoparticles may further be analyzed by fluorescence correlation spectroscopy (FCS) in combination with other methods, including, such as, for example, UV/VIS optical spectroscopy as well as single particle dye bleaching experiments. Analysis by FCS may be used to determine the hydrodynamic size of the nanoparticle and/or number of inorganic nanoparticles per solution volume (i.e., the inorganic nanoparticle concentration). FCS may be performed using a laser. Various lasers may be used based on the dye group(s) being analyzed. Suitable lasers include, but are not limited to, 488 nm solid state lasers (which may be suitable for RhG fluorophores), 543 HeNe lasers (which may be suitable for a TMR fluorophore), a 633 nm solid state laser (which may be suitable for Cy5 and Cy5.5 fluorophores), a 785 nm solid state laser (which may be suitable for dyes such as CW800 and Cy7.5). FCS may also be used in combination with UV/VIS optical spectroscopy as well as single-particle photobleaching experiments to determine the number of dyes per particle.

Various methods of purification and/or analysis may be combined and performed in any order. For example, a plurality of inorganic nanoparticles may first be purified by GPC, analyzed by FCS, and then analyzed by HPLC. In various examples, the purification and analysis may be performed using preparative scale HPLC, analytical scale HPLC, GPC, and the like, and combinations thereof.

Methods of purification and/or analysis may generate eluent containing a purified, an analyzed, and/or a selected portion of inorganic nanoparticles. The purified, analyzed, and/or selected portion of inorganic nanoparticles may be referred to as fractions. The fractions may be combined to generate various compositions comprising desirable combinations of inorganic nanoparticles. For example, a fraction containing a plurality of inorganic nanoparticles, where individual inorganic nanoparticles encapsulate one or more cationic dry groups and/or anionic dye groups, may be combined with a fraction containing a plurality of inorganic nanoparticles, where individual inorganic nanoparticles have one anionic dye group disposed or partially disposed on the exterior surface of the individual inorganic nanoparticles.

In an aspect, the present disclosure provides a method of making inorganic nanoparticles (e.g., ultrasmall nanoparticles). The methods are based on use of aqueous reaction medium (e.g. water). The nanoparticles can be surface functionalized with polyethylene glycol groups (e.g., PEGylated) and/or various dye groups. One or more dye group disposed or partially disposed on the surface of an inorganic nanoparticle may refer to the one or more dye group being disposed on PEG group(s) and/or part of PEG group(s) disposed on the inorganic nanoparticle.

The methods as described herein may be linearly scaled up (e.g., from 10 mL reaction to 1000 mL or greater) without any substantial change in product quality. This scalability may be important for large-scale manufacture of the nanoparticles.

The methods may be carried out in an aqueous reaction medium (e.g., water). For example, the aqueous medium comprises water. Certain reactants may be added to the various reaction mixtures as solutions in a polar aprotic solvent (e.g., DMSO or DMF). In various examples, the aqueous medium does not contain organic solvents (e.g., alcohols such as Ci to C6 alcohols) other than polar aprotic solvents at 10% or greater, 20% or greater, or 30% or greater. In an example, the aqueous medium does not contain alcohols at 1% or greater, 2% or greater, 3% or greater, 4% or greater, or 5% or greater. In an example, the aqueous medium does not contain any detectible alcohols. For example, the reaction media of any of the steps of any of the methods disclosed herein consists essentially of water and, optionally, a polar aprotic solvent.

A method of the present disclosure comprises forming a reaction mixture comprising water, dye precursor, TMOS, base, and PEG-silane. Various mole ratios of the dye precursor, TMOS, base, and PEG-silane may be used. In various examples, the mole ratios of dye precursor, TMOS, base, and PEG-silane are 0.0090-0.032:11-46:0.5-1.5:5-20, including all integer mole ratio values and ranges therebetween (dye precursor:TMOS:base:PEG-silane). For example, if Cy5 is used as a dye precursor, the mole ratio ranges for Cy5:TMOS:base:PEG-silane are 0.0091-0.028:11.4-34:0.5-1.5:5-15, including all mole ratio values and ranges therebetween (e.g., 0.01835:10: 1:10 or 0.009175:11.425:0.5:5 or 0.02725:34.275: 1.5). For example, if Cy5.5 is used as a dye precursor, the mole ratio ranges for Cy5.5:TMOS:base:PEG-silane are 0.01058-0.03176:15.2-45.7:0.5-1.5:6.6-20, including all mole ratio values and ranges therebetween (e.g., 0.021173:30:46:1:13.3 or 0.01058:15.23: 0.5:6.66 or 0.03176:45.7: 1.5:20).

At various points in the methods of the present disclosure, the pH may be adjusted to a desired value or within a desired range. The pH of the reaction mixture can be increased by addition of a base. Examples of suitable bases include ammonium hydroxide and an ammonia in ethanol solution. Additional non-limiting examples of bases include bases capable of forming a quaternary amine (e.g., triethylamine and the like), hydroxide salts of monovalent cations (e.g., NaOH, KOH, and the like), and basic amino acids (e.g., arginine, lysine, and the like). Without intending to be bound by any particular theory, it is considered that hydroxide salts of divalent cations are not suitable bases for a method of the present disclosure.

The concentration of the base (e.g., ammonium hydroxide or ammonia) in the reaction mixture may be 0.001 mM to 60 mM, including every 0.001 mM value and range therebetween (e.g., 0.01 mM to 10 mM, 0.01 mM to 20 mM, 0.01 mM to 30 mM, 0.01 mM to 40 mM, 0.01 mM to 50 mM, 0.01 mM to 60 mM, 0.001 mM to 10 mM, 0.001 mM to 20 mM, 0.001 mM to 30 mM, 0.001 mM to 40 mM, 0.001 mM to 50 mM, 0.001 mM to 60 mM). In various examples, the base is ammonium hydroxide and has a concentration of 0.001 mM to 60 mM, including every 0.001 mM value and range therebetween (e.g., 0.01 mM to 60 mM). In various examples, the concentration of ammonium hydroxide is 0.001 mM to 1 mM, 0.001 mM to 2 mM, 0.001 mM to 2.5 mM, 0.001 mM to 3 mM, 0.001 mM to 4 mM, 0.001 mM to 5 mM, 0.001 to 10 mM, 0.01 mM to 1 mM, 0.01 mM to 2 mM, 0.01 mM to 2.5 mM, 0.01 mM to 3 mM, 0.01 mM to 4 mM, 0.01 mM to 5 mM, 0.01 to 10 mM, 0.1 mM to 1 mM, 0.1 mM to 2 mM, 0.1 mM to 2.5 mM, 0.1 mM to 3 mM, 0.1 mM to 4 mM, 0.1 mM to 5 mM, 0.1 to 10 mM. In various examples, the base is ammonia in ethanol and has a concentration of 0.01 mM to 60 mM, including every 0.001 mM value and range therebetween. In various examples, the concentration of ammonia in ethanol is 0.01 mM to 1 mM, 0.01 mM to 2 mM, 0.01 mM to 2.5 mM, 0.01 mM to 3 mM, 0.01 mM to 4 mM, 0.01 mM to 5 mM, 0.01 to 10 mM, 0.1 mM to 1 mM, 0.1 mM to 2 mM, 0.1 mM to 2.5 mM, 0.1 mM to 3 mM, 0.1 mM to 4 mM, 0.1 mM to 5 mM, 0.1 to 10 mM.

In various examples, when using Cy5 as the dye precursor, the reaction mixture comprises 0.367 μmol Cy5, 457 μmol TMOS, 200 μmol PEG-silane, and 20 μmol base (e.g., ammonium hydroxide) and 10 mL of water. In various examples, when using Cy5.5 as the dye precursor, the reaction mixture comprises 0.3176 μmol, 457 μmol TMOS, 200 μmol PEG-silane, and 15 μmol base and 10 mL water.

Without intending to be bound by any particular theory, it is considered that base concentration (e.g., ammonium hydroxide concentration) is a parameter to control the surface chemistry heterogeneity. It is considered that the base (e.g., ammonium hydroxide) controls the rate of hydrolysis, condensation, and surface charge of the silica clusters formed. For example, if ammonium hydroxide concentration is too low, the primary silica clusters will be prone to aggregation and the size will increase due to uncontrolled aggregation of the silica clusters. For example, if ammonium hydroxide concentration is too high, a dye or plurality of dyes will not be fully encapsulated.

In various examples, the concentration of base (e.g., ammonium hydroxide) is optimized for a particular dye used.

For example, a method of making inorganic nanoparticles functionalized with polyethylene glycol groups (i.e., PEGylated inorganic nanoparticles) and dye molecules comprises: a) forming a reaction mixture at room temperature (e.g., 15° C. to 25° C. depending on the location) comprising water, a silica core forming monomer (e.g., TMOS) (e.g., at a concentration of 11 mM to 270 mM), and one or more dye group precursor, wherein the pH of the reaction mixture (which can be adjusted using a base such as, for example, ammonium hydroxide) is 6 to 11 (which results in formation of core precursor nanoparticles having an average size (e.g., longest dimension) of, for example, 1 nm to 2 nm) (e.g., a pH of 6 to 9); b) either i) holding the reaction mixture at a time (t¹) and temperature (T¹) (e.g., (t¹) 0.5 hours to 7 days (e.g., 0.5 days to 7 days) at room temperature to 95° C. (T¹), and in various examples, the T¹ is 0.5 hours to 2 hours), whereby nanoparticles (core nanoparticles) having an average size (e.g., longest dimension) of 2 to 15 nm are formed, or ii) cooling the reaction mixture to room temperature, if necessary, and adding a shell forming monomer (e.g., tetraethyl orthosilicates, other than TMOS, such as, for example, TEOS or TPOS) (the addition is carried out such that the shell forming monomer concentration is below the threshold for secondary nucleation) to the reaction mixture from a), whereby inorganic nanoparticles having an average size (e.g., longest dimension) of 2 to 50 nm (e.g., 2 to 15 nm) are formed; c) adjusting, if necessary, the pH of the reaction mixture to a pH of 6 to 10 comprising the inorganic nanoparticles from b) i) or b) ii), respectively; and d) optionally (PEGylating the inorganic nanoparticles by) adding at room temperature to the reaction mixture comprising the inorganic nanoparticles from b) i) or b) ii), respectively, a PEG-silane conjugate (comprising a PEG moiety covalently bound to a silane moiety) (e.g., at a concentration of 10 mM to 60 mM) (e.g., PEG-silane conjugate dissolved in a polar aprotic solvent such as, for example, DMSO or DMF) and holding the resulting reaction mixture at a time (t²) and temperature (T²) (e.g., (t²) 0.5 minutes to 24 hours at room temperature (T²)) (whereby at least a portion of the PEG-silane conjugate molecules are adsorbed on at least a portion of the surface of the core nanoparticles or core-shell nanoparticles from b)); e) heating the mixture from d) at a time (t³) and temperature (T³) (e.g., (t³) 1 hour to 24 hours at 40° C. to 100° C. (T³)), whereby the inorganic nanoparticles functionalized with one or more dye groups and surface functionalized with polyethylene glycol groups are formed; and f) purifying the reaction mixture containing the inorganic nanoparticles functionalized with one or more dye groups and surface functionalized with polyethylene glycol groups by liquid chromatography, where the mole ratio of dye precursor, TMOS, base, and PEG-silane is 0.0090-0.032:11-46:0.5-1.5:5-20, including all integer mole ratio values and ranges therebetween (dye precursor:TMOS:base:PEG-silane). In various examples, the core is formed with about one hour (e.g., one hour).

The inorganic nanoparticles may be subjected to post-synthesis processing steps. For example, after synthesis (e.g., after e) in the example above) the solution is cooled to room temperature and then transferred into a dialysis membrane tube (e.g., a dialysis membrane tube having a Molecular Weight Cut off 10,000, which are commercially available (e.g., from Pierce)). The solution in the dialysis tube is dialyzed in DI-water (volume of water is 200 times more than the reaction volume, e.g., 2000 mL water for a 10 mL reaction) and the water is changed every day for one to six days to wash away remaining reagents, e.g., ammonium hydroxide and free silane molecules. The particles are then filtered through a 200 nm syringe filter (fisher brand) to remove aggregates or dust. If desired, additional purification processes, including gel permeation chromatography and high-performance liquid chromatography, can be applied to the nanoparticles to further ensure the high purify of the synthesized particles (e.g., 1% or less unreacted reagents or aggregates). After any purification processes, the purified nanoparticles can be transferred back to deionized water if other solvent is used in the additional processes.

The cores can be silica cores. The reaction mixture used in silica core formation can comprise TMOS as the only silica core forming monomer.

The cores can be aluminosilicate cores. The reaction mixture used in aluminosilicate core formation can comprise TMOS as the only silica core forming monomer and one or more alumina core forming monomer (e.g., an aluminum alkoxide such as, for example, aluminum-tri-sec-butoxide or a combination of aluminum alkoxides).

In the case of aluminosilicate core synthesis, the pH of the reaction mixture is adjusted to a pH of 1 to 2 prior to addition of the alumina core forming monomer. After aluminosilicate core formation, the pH of the solution is adjusted to a pH of 7 to 9 and, optionally, PEG with molecular weight between 100 and 1,000 g/mol, including all integer values and ranges therebetween, at concentration of 10 mM to 75 mM, including all integer mM values and ranges therebetween, is added to the reaction mixture prior to adjusting the pH of the reaction mixture to a pH of 7 to 9.

The reaction mixture used to form inorganic nanoparticles can also comprise a dye precursor (e.g., a positively charged dye precursor). In this case, the resulting core or core-shell nanoparticles have one or more dye molecules (e.g., positively charged dye molecules) encapsulated or incorporated therein. For example, core nanoparticle has 1, 2, 3, 4, 5, 6, or 7 positively charged dye molecules encapsulated therein. Mixtures of dye precursors can be used. The dye precursor (e.g., positively charged dye precursor) may be a dye (e.g., positively charged dye) conjugated to a silane. For example, a positively charged dye with maleimido functionality is conjugated to thiol-functionalized silane. In another example, a positively charged dye with NHS ester functionality is conjugated to amine-functionalized silane. Examples of suitable silanes and conjugation chemistries are known in the art. The dye can have an emission (e.g., fluorescence) wavelength of 400 nm (blue) to 900 nm (near-infrared). For example, the dye is a near infrared (NIR) dye. Examples of suitable dyes include, but are not limited to, rhodamine green (RHG), tetramethylrhodamine (TMR), Cyanine 5 (Cy5), Cyanine 5.5 (Cy5.5), Cyanine 7 (Cy7), ATTO425, ATTO647N, ATTO647, ATTO680, Dyomics DY800, Dyomics DY782 and IRDye 800CW, and the inorganic nanoparticles surface functionalized with polyethylene glycol groups may have one or more fluorescent positively charged dye molecules encapsulated therein. Examples of dyes include negatively charged dyes, such as, for example, sulfo-Cy5.5, sulfo-Cy5, sulfo-Cy3, Alexa Fluor 532, Alexa Fluor 430, ATTO430LS, ATTO488, ATTO490LS, ATTO532, ATTO594, and the like, and combinations thereof; net neutral dyes, such as, for example, tetramethylrhodamine (TMR), ATTO390, ATTO425, ATTO565, ATTO590, ATTO647, ATTO650, ATTO655, ATTO680, ATTO700, and the like, and combinations thereof, and positively charged dyes, such as, for example, Cy5.5, Cy5, Cy3, ATTO647N, methylene blue, ATTO663, ATTO620, ATTO665, ATTO465, ATTO495, ATTO520, ATTORho6G, ATTORho3B, ATTORho11, ATTORho12, ATTOThio12, ATTO580Q, ATTORho101, ATTORho13, ATTO610, ATTO612Q, ATTO647N, ATTORho14, ATTOOxa12, ATTO725, ATTO740, ATTOMB2, and the like, and combinations thereof. The dyes may have functional groups suitable for conjugation chemistry, such as, for example, carboxylic acids, NHS-esters, and the like, and may be referred to as such. In an illustrative example, Cy5-NHS-ester is the NHS ester of Cy5. The dye groups may be covalently bound to the silica matrix or aluminosilicate matrix of the inorganic nanoparticle and/or covalently bound to an exterior surface of the inorganic nanoparticle and/or are part of a PEG group.

A silica shell may be formed on core nanoparticles. The silica shell is formed after, for example, core formation is complete. Examples of silica shell forming precursors include tetraalkylorthosilicates such as, for example, TEOS and TPOS. Mixtures of silica shell forming precursors can be used. TMOS is not a silica shell forming precursor. The silica shell forming precursor can be added to the reaction mixture as a solution in a polar aprotic solvent. Examples, of suitable polar aprotic solvents include DMSO and DMF.

It is desirable to add the silica shell forming precursors in separate aliquots. For example, the shell forming monomer(s) is/are added in separate aliquots (e.g., 40 to 500 aliquots, including every integer aliquot value and range therebetween) The aliquots can include one or more shell forming precursor (e.g., TEOS and/or TPOS) and a polar aprotic solvent (e.g., DMSO). Each aliquot may have 1 to 20 micromoles of shell forming monomer, including every 0.1 micromole value and range therebetween. The interval between aliquot addition may be 1 to 60 minutes, including all integer second values and ranges therebetween. The pH of the reaction mixture can vary during the silica shell forming process. It is desirable to adjust the pH to maintain a pH of 7-8.

After inorganic nanoparticle formation, the inorganic nanoparticle can by reacted with one or more PEG-silane conjugates. Various PEG-silane conjugates can be added together or in various orders. This process is also referred to herein as PEGylation. The conversion percentage of PEG-silane is between 5% and 40% and the polyethylene glycol surface density is 1.3 to 2.1 polyethylene glycol molecules per nm². The conversion percentage of ligand-functionalized PEG-silane is 40% to 100% and the number of ligand-functionalized PEG-silane precursors reacted with each particle is 3 to 90.

PEGylation can be carried out at a variety of times and temperatures. For example, in the case of inorganic nanoparticles, PEGylation can be carried out by contacting the nanoparticles at room temperature for 0.5 minutes to 24 hours (e.g., overnight). For example, in the case of aluminosilicate nanoparticles (e.g., aluminosilicate core nanoparticles or inorganic nanoparticle) the temperature is 80° C. overnight.

The chain length of the PEG moiety of the PEG-silane (i.e., the molecular weight of the PEG moiety) can be tuned from 3 to 24 ethylene glycol monomers (e.g., 3 to 6, 3 to 9, 6 to 9, 8 to 12, or 8 to 24 ethylene glycol monomers). The PEG chain length of PEG-silane can be selected to tune the thickness of the PEG layer surrounding the particle and the pharmacokinetics (PK) and biodistributrion profiles of the PEGylated particles. The PEG chain length of ligand-functionalized PEG-silane can be used to tune the accessibility of the ligand groups on the surface of the PEG layer of the particles resulting in varying binding and targeting performance.

PEG-silane conjugates can comprise a ligand. The ligand is covalently bound to the PEG moiety of the PEG-silane conjugates. The ligand can be conjugated to a terminus of the PEG moiety opposite the terminus conjugated to the silane moiety. The PEG-silane conjugate can be formed using a heterobifunctional PEG compound (e.g., maleimido-functionalized heterobifunctional PEGs, NHS ester-functionalized heterobifunctional PEGs, amine-functionalized heterobifunctional PEGs, thiol-functionalized heterobifunctional PEGs, etc.). Examples of suitable ligands include, but are not limited to, peptides (natural or synthetic), cyclic peptides, ligands comprising a radio label (e.g., ¹²⁴I, ¹³¹I, ²²⁵Ac, or ¹⁷⁷Lu), antibodies, antibody fragments, DNA, RNA, simple sugars, oligosaccharides, drug molecules (e.g. small molecule inhibitors, toxic drugs), and ligands comprising a reactive group (e.g., a reactive group that can be conjugated to a molecule such a drug molecule, gefitinib, etc.).

For additional particle functionalization (e.g., to generate multifunctional nanoparticles) amine- and/or thiol-functionalized silane molecules may be inserted between PEG chains and onto the silica surface of inorganic nanoparticles (e.g., C′ dots), to which additional functional ligands (e.g., sensor dye molecules, additional chelators for radiometals, or additional functional groups in order to add pharmaceutical compounds) can subsequently be attached. This post-PEGylation surface modification by insertion (PPSMI) approach only requires a few extra steps sandwiched between nanoparticle (e.g., C′ dot) PEGylation and purification in a one-pot type water-based synthesis without diminishing high quality NP generation. The resulting nanoparticles (e.g., C′ dots) with additional functionalities exhibit physico-chemical properties like their size and PEG density close to clinically translated nanoparticles (e.g., C dots), opening a gate to the diversification of their clinical applications. Modification of a nanoparticle synthesis (e.g., a C′ dot synthesis) enables, for example, large numbers of targeting peptides per particle, as well as a facile and versatile spectroscopic approach to quantitatively assess the specific numbers of the different surface ligands by deconvolution of absorption spectra into individual components.

For example, PEG-silane conjugate comprising a ligand is added in addition to PEG-silane (e.g., in d) in the example above). In this case, inorganic nanoparticles surface functionalized with polyethylene glycol groups and polyethylene groups comprising a ligand are formed. The conversion percentage of ligand-functionalized or reactive group-functionalized PEG-silane is 40% to 100% and the number of ligand-functionalized PEG-silane precursors reacted with each particle is 3 to 600.

For example, before or after (e.g., 20 seconds to 5 minutes before or after) the PEG-silane conjugate is added (e.g., in d) in the example above) a PEG-silane conjugate comprising a ligand (e.g., at concentration between 0.05 mM and 2.5 mM) is added at room temperature to the reaction mixture comprising the inorganic nanoparticles (e.g., from b) i) or b) ii), respectively, in the example above). The resulting reaction mixture is held at a time (t⁴) and temperature (T⁴) (e.g., (t⁴) 0.5 minutes to 24 hours at room temperature (T⁴)), where at least a portion of the PEG-silane conjugate molecules are adsorbed on at least a portion of the surface of the core nanoparticles or core-shell nanoparticles (e.g., from b) in the example above). Subsequently, the reaction mixture is heated at a time (t⁵) and temperature (T⁵) (e.g., (t⁵) 1 hour to 24 hours at 40° C. to 100° C. (T⁵)), where inorganic nanoparticles surface functionalized with polyethylene glycol groups comprising a ligand are formed. Optionally, subsequently adding at room temperature to the resulting reaction mixture comprising inorganic nanoparticles surface functionalized with polyethylene glycol groups comprising a ligand and a PEG-silane conjugate (the concentration of PEG-silane no ligand is between 10 mM and 75 mM) (e.g., PEG-silane conjugate dissolved in a polar aprotic solvent such as, for example, DMSO or DMF), holding the resulting reaction mixture at a time (t⁶) and temperature (T⁶) (e.g., (t⁶) 0.5 minutes to 24 hours at room temperature (T⁶)) (whereby at least a portion of the PEG-silane conjugate molecules are adsorbed on at least a portion of the surface of the inorganic nanoparticles surface functionalized with polyethylene glycol groups comprising a ligand, and heating the resulting mixture from at a time (t⁷) and temperature (T⁷) (e.g., (t⁷) 1 hour to 24 hours at 40° C. to 100° C. (T⁷)), whereby inorganic nanoparticles surface functionalized with polyethylene glycol groups comprising a ligand are formed.

In another example, at least a portion of or all of the PEG-silane has a reactive group on a terminus of the PEG moiety opposite the terminus conjugated to the silane moiety of the PEG-silane conjugate (is formed from a heterobifunctional PEG compound) and after formation of the inorganic nanoparticles surface functionalized with polyethylene glycol groups having a reactive group, and, optionally, polyethylene glycol groups. Optionally, polyethylene glycol groups are reacted with a second ligand (which can be the same or different than the ligand of the inorganic nanoparticles surface functionalized with polyethylene glycol groups and polyethylene glycol group comprising a ligand) functionalized with a second reactive group (which can be the same or different than the reactive group of the inorganic nanoparticles surface functionalized with polyethylene glycol groups and polyethylene glycol group comprising a ligand) thereby forming inorganic nanoparticles surface functionalized with polyethylene groups functionalized with a second ligand and, optionally, polyethylene glycol groups.

In another example, at least a portion of or all of the PEG-silane has a reactive group on a terminus of the PEG moiety opposite the terminus conjugated to the silane moiety of the PEG-silane conjugate (is formed from a heterobifunctional PEG compound) and after formation of the inorganic nanoparticles surface functionalized with polyethylene glycol groups and, optionally having a reactive group, and, optionally, polyethylene glycol groups, are reacted with a second ligand (which can be the same or different than the ligand of the inorganic nanoparticles surface functionalized with polyethylene glycol groups and polyethylene glycol group comprising a ligand) functionalized with a second reactive group (which can be the same or different than the reactive group of the inorganic nanoparticles surface functionalized with polyethylene glycol groups and polyethylene glycol group comprising a ligand) thereby forming inorganic nanoparticles surface functionalized with polyethylene groups functionalized with a second ligand and, optionally, polyethylene glycol groups, where at least a portion of the PEG-silane has a reactive group on a terminus of the PEG moiety opposite the terminus conjugated to the silane moiety of the PEG-silane conjugate (is formed from a heterobifunctional PEG compound) and after formation of the inorganic nanoparticles surface functionalized with polyethylene glycol groups having a reactive group, inorganic nanoparticles surface functionalized with polyethylene glycol groups having a reactive group and polyethylene glycol groups comprising a ligand, the reactive group are reacted with a second ligand functionalized with a reactive group (which can be the same or different than the ligand of the inorganic nanoparticles surface functionalized with polyethylene glycol groups and polyethylene glycol group comprising a ligand) thereby forming inorganic nanoparticles surface functionalized with polyethylene glycol groups and polyethylene groups functionalized with a second ligand or inorganic nanoparticles surface functionalized with polyethylene glycol groups comprising a ligand that is functionalized with the second ligand.

The inorganic nanoparticles with PEG groups functionalized with reactive groups can be further functionalized with one or more ligands. For example, a functionalized ligand can be reacted with a reactive group of a PEG group. Examples of suitable reaction chemistries and conditions for post-nanoparticle synthesis functionalization are known in the art.

The inorganic nanoparticles may have a narrow size distribution. In various examples, the nanoparticle size distribution (before or after PEGylation), not including extraneous materials such as, for example, unreacted reagents, dust particles/aggregates, is +/−5, 10, 15, or 20% of the average particle size (e.g., longest dimension). The particle size may be determined by methods known in the art. For example, the particle size is determined by TEM, GPS, or DLS. DLS contains systematic deviation and, therefore, the DLS size distribution may not correlate with the size distribution determined by TEM or GPS.

In an aspect, the present disclosure provides compositions comprising inorganic nanoparticles of the present disclosure. The compositions can comprise one or more types (e.g., having different average size and/or one or more different compositional feature).

For example, a composition comprises a plurality of inorganic nanoparticles (e.g., silica core nanoparticles, silica core-shell nanoparticles, aluminosilicate core nanoparticles, aluminosilicate core-shell nanoparticles). Any of the inorganic nanoparticles may be surface functionalized with one or more type of polyethylene glycol groups (e.g., polyethylene glycol groups, functionalized (e.g., functionalized with one or more ligand and/or a reactive group) polyethylene glycol groups, or a combination thereof). Any of the inorganic nanoparticles can have a dye group or combination of dye groups (e.g., a NIR dye, such as, for example, a positively charged NIR dye) encapsulated therein. The dye groups are covalently bound to the inorganic nanoparticles. The inorganic nanoparticles can be made by a method of the present disclosure. For example, location of the dye in and/or on the inorganic nanoparticles can be determined by the charge of the dye. The dye groups may be positively charged, negatively charged, or be net neutral.

Dye groups that are fully encapsulated in the inorganic nanoparticle remain encapsulated in inorganic nanoparticle such that no free dye leaches into the aqueous medium suspending the nanoparticles. Dye groups remain encapsulated for periods up to 6 months to 2 years (e.g., 6 months, 9 months, 12 months, 18 months, or 24 months). For example, an aqueous (e.g., water) composition comprising inorganic nanoparticles with positively charged dyes are stable for a period of up to 6 months to 2 years (e.g., 6 months, 9 months, 12 months, 18 months, or 24 months) and do not exhibit observable free dye in the aqueous medium (e.g., water) during this period. For example, the composition does not exhibit observable free dye by HPLC (e.g., an HPLC method described herein) in the aqueous medium (e.g., water) during this period.

The inorganic nanoparticles in a composition can have a variety of sizes. The inorganic nanoparticles can have a core size of 2 to 50 nm (e.g., 2 to 10 nm or 2 to 5 nm), including all 0.1 nm values and ranges therebetween. In various examples, the inorganic nanoparticles have a core size of 2, 2.5, 3, 3.5, 4, 4.5, 5, 5.5, 6, 6.5, 7, 7.5, 8, 8.5, 9, 9.5, 9.99, 10, 10.5, 11, 11.5, 12, 12.5, 13, 13.5, 14, 14.5, or 15 nm. In various examples, at least 90%, 95%, 96%, 97%, 98%, 99%, 99.5% 99.9%, or 100% of the inorganic nanoparticles have a size (e.g., longest dimension) of 2 to 50 nm (e.g., 2 to 10 nm or 2 to 5 nm). In various examples, at least 90%, 95%, 96%, 97%, 98%, 99%, 99.5% 99.9%, or 100% of the inorganic nanoparticles have a size (e.g., longest dimension) of 2 to 50 nm. For the exemplary size distributions, the composition may not be subjected to any particle-size discriminating (particle size selection/removal) processes (e.g., filtration, dialysis, chromatography (e.g., GPC), centrifugation, etc.). For example, the inorganic nanoparticles of the present disclosure are the only inorganic nanoparticles in the composition. In an example, an inorganic nanoparticle may have 0-4 shells (e.g., 0, 1, 2, 3, or 4).

The inorganic nanoparticles in a composition can have a variety of sizes. The inorganic nanoparticles can have a core size of 2 to 15 nm (e.g., 2 to 10 nm or 2 to 9.99 nm), including all 0.1 nm values and ranges therebetween. In various examples, the inorganic nanoparticles have a core size of 2, 2.5, 3, 3.5, 4, 4.5, 5, 5.5, 6, 6.5, 7, 7.5, 8, 8.5, 9, 9.5, 9.99, 10, 10.5, 11, 11.5, 12, 12.5, 13, 13.5, 14, 14.5, or 15 nm. In various examples, at least 90%, 95%, 96%, 97%, 98%, 99%, 99.5% 99.9%, or 100% of the inorganic nanoparticles have a size (e.g., longest dimension) of 2 to 15 nm (e.g., 2 to 10 nm or 2 to 9.99 nm). In various examples, at least 90%, 95%, 96%, 97%, 98%, 99%, 99.5% 99.9%, or 100% of the core-shell nanoparticles have a size (e.g., longest dimension) of 2 to 50 nm. For the exemplary size distributions, the composition may not be subjected to any particle-size discriminating (particle size selection/removal) processes (e.g., filtration, dialysis, chromatography (e.g., GPC), centrifugation, etc.). For example, the inorganic nanoparticles of the present disclosure are the only inorganic nanoparticles in the composition. In an example, an inorganic nanoparticle may have 0-4 shells (e.g., 0, 1, 2, 3, or 4).

The composition can comprise additional components. For example, the composition can also comprise a buffer suitable for administration to an individual (e.g., a mammal such as, for example, a human). The buffer may be a pharmaceutically acceptable carrier.

The compositions, as synthesized and before any post-synthesis processing/treatment, can have inorganic nanoparticles, particles (2-15 nm, e.g., 2-10 nm, e.g., 2 to 10 nm or 2 to 5 nm), dust particles/aggregates (>20 nm), unreacted reagents (<2 nm).

In various examples, the as-synthesized produced nanoparticles are not subjected to any post-synthesis purificaiton process(es) (e.g., other than isolation from the reaction mixture). Non-limiting examples of purification process(es) include chromatography (e.g., size exclusion chromatography (SEC) and the like), reprecipitation, salt exchange, solvent extraction, and the like, and combinations thereof. In various examples, purification includes separation of one or more undesired materials, components, products of the method, or the like, or a combination thereof.

A composition may comprise a plurality of inorganic nanoparticles, where the individual inorganic nanoparticles of the plurality of inorganic nanoparticles comprise 1-7 positively charged dye group(s), where: i) none of the positively charged dye groups are disposed or partially disposed on the surface of the inorganic nanoparticles; or ii) the majority (e.g., greater than 50%) of the inorganic nanoparticles have at least one (e.g., one, two, three, four, five, six, seven, or a combination thereof) positively charged dye group(s) disposed or partially disposed on the surface of the inorganic nanoparticles; or iii) all of the positively charged dye groups are disposed or partially disposed on the surface of the inorganic nanoparticles.

In an example, a composition comprising a plurality of inorganic nanoparticles, where the individual inorganic nanoparticles of the plurality of inorganic nanoparticles comprise 1-7 positively charged dye group(s) may consist essentially of individual inorganic nanoparticles having 0, 1, 2, 3, 4, 5, 6, or 7 dye groups fully encapsulated in the inorganic nanoparticles.

A composition may comprise a plurality of inorganic nanoparticles, where the individual inorganic nanoparticles of the plurality of inorganic nanoparticles comprise 1-7 net neutral dye group(s), where: i) none of the net neutral dye groups are disposed or partially disposed on the surface of the inorganic nanoparticles; or ii) the majority (e.g., greater than 50%) of the inorganic nanoparticles have at least one (e.g., one, two, three, four, five, six, seven, or a combination thereof) net neutral dye group(s) disposed or partially disposed on the surface of the inorganic nanoparticles; or iii) all of the net neutral dye groups are disposed or partially disposed on the surface of the inorganic nanoparticles.

In an example, a composition comprising a plurality of inorganic nanoparticles, where the individual inorganic nanoparticles of the plurality of inorganic nanoparticles comprise 1-7 net neutral dye group(s) may consist essentially of individual inorganic nanoparticles having 0, 1, 2, 3, 4, 5, 6, or 7 dye groups fully encapsulated in the inorganic nanoparticles.

A composition comprising a plurality of inorganic nanoparticles, where the individual inorganic nanoparticles of the plurality of inorganic nanoparticles comprise 1-7 negatively charged dye groups, where: i) none of the negatively charged dye groups are disposed or partially disposed on the surface of the inorganic nanoparticles; or ii) the majority (e.g., greater than 50%) of the inorganic nanoparticles have none of the negatively charged dye groups are disposed or partially disposed on the surface of the inorganic nanoparticles; or iii) the majority of the inorganic nanoparticles have 1, 2, 3, 4, 5, 6, or 7 of the negatively charged dye group(s) are disposed or partially disposed on the surface of the inorganic nanoparticles.

In an example, a composition comprising a plurality of inorganic nanoparticles, where the individual inorganic nanoparticles of the plurality of inorganic nanoparticles comprise 1-7 negatively charged dye group(s) may consist essentially of individual inorganic nanoparticles having 0, 1, 2, 3, 4, 5, 6, or 7 negatively charged dye groups fully encapsulated in the inorganic nanoparticles.

In an example, where one or more dye group is positively charged or has net neutral charge and the plurality of inorganic nanoparticles do not exhibit size-dependent surface inhomogeneity, where the size-dependent surface inhomogeneity is determined by HPLC.

In an aspect, the present disclosure provides uses of the inorganic nanoparticles and compositions of the present disclosure. For example, inorganic nanoparticles or a composition comprising the inorganic nanoparticles are used in delivery and/or imaging methods.

The ligands carried by the inorganic nanoparticles can include diagnostic and/or therapeutic agents (e.g., drugs). Examples of therapeutic agents include, but are not limited to, chemotherapeutic agents, antibiotics, antifungal agents, antiparasitic agents, antiviral agents, and combinations thereof. An affinity ligand may be also be conjugated to the nanoparticle to allow targeted delivery of the nanoparticles. For example, the inorganic nanoparticles may be conjugated to a ligand which is capable of binding to a cellular component (e.g., on the cell membrane or in the intracellular compartment) associated with a specific cell type. The targeted molecule can be a tumor marker or a molecule in a signaling pathway. The ligand can have specific binding affinity to certain cell types, such as, for example, tumor cells. In certain examples, the ligand may be used for guiding the nanoparticles to specific areas, such as, for example, liver, spleen, brain, or the like. Imaging can be used to determine the location of the nanoparticles in an individual.

The inorganic nanoparticles or compositions comprising inorganic nanoparticles can be administered to individuals for example, in pharmaceutically-acceptable carriers, which facilitate transporting the inorganic nanoparticles from one organ or portion of the body to another organ or portion of the body. Examples of individuals include animals such as human and non-human animals. Examples of individuals also include mammals.

Pharmaceutically acceptable carriers are generally aqueous based. Some examples of materials which can be used in pharmaceutically-acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. (See REMINGTON'S PHARM. SCI., 22th Ed. (Mack Publ. Co., Easton (2012)).

Compositions comprising the present inorganic nanoparticles can be administered to an individual by any suitable route—either alone or as in combination with other agents. Administration can be accomplished by any means, such as, for example, by parenteral, mucosal, pulmonary, topical, catheter-based, or oral means of delivery. Parenteral delivery can include, for example, subcutaneous, intravenous, intramuscular, intra-arterial, and injection into the tissue of an organ. Mucosal delivery can include, for example, intranasal delivery. Pulmonary delivery can include inhalation of the agent. Catheter-based delivery can include delivery by iontophoretic catheter-based delivery. Oral delivery can include delivery of an enteric-coated pill or administration of a liquid by mouth. Transdermal delivery can include delivery via the use of dermal patches.

Following administration of a composition comprising the present inorganic nanoparticles, the path, location, and clearance of the inorganic nanoparticles can be monitored using one or more imaging techniques. Examples of suitable imaging techniques include Artemis Fluorescence Camera System.

This disclosure provides a method for imaging biological material such as cells, extracellular components, or tissues comprising contacting the biological material with inorganic nanoparticles comprising one or more positively charged dyes, or compositions comprising the nanoparticles; directing excitation electromagnetic (e/m) radiation, such as light, on to the tissues or cells thereby exciting the positively charged dye molecules; detecting e/m radiation emitted by the excited positively charged dye molecules; and capturing and processing the detected e/m radiation to provide one or more images of the biological material. One or more of these steps can be carried out in vitro or in vivo. For example, the cells or tissues can be present in an individual or can be present in culture. Exposure of cells or tissues to e/m radiation can be effected in vitro (e.g., under culture conditions) or can be effected in vivo. For directing e/m radiation at cells, extracellular materials, tissues, organs and the like within an individual or any portion of an individual's body that are not easily accessible, fiber optical instruments can be used.

For example, a method for imaging of a region within an individual comprises (a) administering to the individual inorganic nanoparticles or a composition of the present disclosure comprising one or more positively charged dye molecules; (b) directing excitation light into the subject, thereby exciting at least one of the one or more positively charged dye molecules; (c) detecting excited light, the detected light having been emitted by said positively charged dye molecules in the individuals as a result of excitation by the excitation light; and (d) processing signals corresponding to the detected light to provide one or more images (e.g. a real-time video stream) of the region within the subject.

Since the fluorescent particles are brighter than free dye, fluorescent particles can be used for tissue imaging, as well as to image the metastasis tumor. Additionally or alternatively, radioisotopes can be further attached to the ligand groups (e.g., tyrosine residue or chelator) of the ligand-functionalized particles or to the silica matrix of the PEGylated particles without specific ligand functionalization for photoinduced electron transfer imaging. If the radioisotopes are chosen to be therapeutic, such as ²²⁵Ac or ¹⁷⁷Lu, this in turn would result in particles with additional radiotherapeutic properties.

For example, drug-linker conjugate, where the linker group can be specifically cleaved by enzyme or acid condition in tumor for drug release, can be covalently attached to the functional ligands on the particles for drug delivery. For example, drug-linker-thiol conjugates can be attached to maleimido-PEG-particles through thiol-maleimido conjugation reaction post the synthesis of maleimido-PEG-particles. Additionally, both drug-linker conjugate and cancer targeting peptides can be attached to the particle surface for drug delivery specifically to tumor.

The steps of the method described in the various embodiments and examples disclosed herein are sufficient to carry out the methods and produce the compositions of the present disclosure. Thus, in an embodiment, the method consists essentially of a combination of the steps of the methods disclosed herein. In another embodiment, the method consists of such steps.

The following Statements describe various embodiments of the present disclosure.

Statement 1. A method for synthesizing an inorganic nanoparticle comprising one or more dyes and surface functionalized with polyethylene glycol (PEG) groups, comprising a) forming a reaction mixture at room temperature comprising water, TMOS, and a dye precursor, wherein the pH of the reaction mixture is 6 to 11 (e.g., 6 to 9); b) either i) holding the reaction mixture at a time (t¹) and temperature (T′), whereby inorganic nanoparticles having an average size of 2 to 15 nm are formed, or ii) cooling the reaction mixture to room temperature, if necessary, and adding a shell forming monomer to the reaction mixture from a), whereby inorganic nanoparticles have a core size of 2 to 15 nm and/or an average size of 2 to 50 nm are formed; c) adjusting, if necessary, the pH of the reaction mixture comprising the inorganic nanoparticles from b) i) or b) ii) to a pH of 6 to 10; d) adding at room temperature to the reaction mixture comprising the inorganic nanoparticles from b) i) or b) ii), respectively, a PEG-silane conjugate and holding the resulting reaction mixture at a time (t²) and temperature (T²); e) heating the mixture from d) at a time (t³) and temperature (T), whereby the inorganic nanoparticles surface functionalized with PEG groups are formed; f) purifying the reaction mixture by liquid chromatography. In other examples, a method for synthesizing an inorganic nanoparticle comprising one or more dyes and surface functionalized with polyethylene glycol (PEG) groups, comprises a) forming a reaction mixture at room temperature comprising water, TMOS, a base, and a dye precursor; b) either i) holding the reaction mixture at a time (t¹) and temperature (T¹), whereby inorganic nanoparticles having an average size of 2 to 15 nm are formed, or ii) cooling the reaction mixture to room temperature, if necessary, and adding a shell forming monomer to the reaction mixture from a), whereby inorganic nanoparticles have a core size of 2 to 15 nm and/or an average size of 2 to 50 nm are formed; c) adjusting, if necessary, the pH of the reaction mixture comprising the inorganic nanoparticles from b) i) or b) ii) to a pH of 6 to 10; d) adding at room temperature to the reaction mixture comprising the inorganic nanoparticles from b) i) or b) ii) a PEG-silane conjugate and holding the resulting reaction mixture at a time (t²) and temperature (T²); e) heating the mixture from d) at a time (t³) and temperature (T), whereby the inorganic nanoparticles surface functionalized with PEG groups are formed; f) purifying the reaction mixture by liquid chromatography, wherein the mole ratio of the dye precursor, TMOS, the base, and the PEG-silane is 0.0090-0.032:11-46:0.5-1.5:5-20, and the method yields the inorganic nanoparticle comprising one or more dyes and surface functionlized with polyethylene glycol (PEG) groups. The one or more dyes are fully encapsulated in the inorganic nanoparticle.

Statement 2. A method of Statement 1, where the base chosen from ammonium hydroxide, ammonia in ethanol, triethyl amine, sodium hydroxide, potassium hydroxide, and the like, combinations thereof.

Statement 3. A method of Statement 1 or Statement 2, where the base has a concentration and the concentration is 0.001 mM to 60 mM, including every 0.001 mM value and range therebetween (0.01 mM to 1 mM, 0.01 mM to 2 mM, 0.01 mM to 2.5 mM, 0.01 mM to 3 mM, 0.01 mM to 4 mM, 0.01 mM to 5 mM, 0.01 to 10 mM, 0.1 mM to 1 mM, 0.1 mM to 2 mM, 0.1 mM to 2.5 mM, 0.1 mM to 3 mM, 0.1 mM to 4 mM, 0.1 mM to 5 mM, 0.1 to 10 mM, 0.001 mM to 1 mM, 0.001 mM to 2 mM, 0.001 mM to 2.5 mM, 0.001 mM to 3 mM, 0.001 mM to 4 mM, 0.001 mM to 5 mM, 0.001 to 10 mM).

Statement 4. A method of any one of the preceding Statements, where purifying comprises isolating a selected portion of a plurality of inorganic nanoparticles from the reaction mixture.

Statement 5. A method of any one of the preceding Statements, further comprising analyzing the selected portion of the plurality of inorganic nanoparticles via GPC.

Statement 6. A method of any one of the preceding Statements, further comprising analyzing the selected portion of the plurality of inorganic nanoparticles via HPLC.

Statement 7. A method of any one of the preceding Statements, where the purification step comprises: depositing a plurality of inorganic nanoparticles in a chromatography column comprising an input in fluid communication with a stationary phase in fluid communication with an output in fluid communication with a detector; passing a mobile phase through the chromatography column, such that the plurality of inorganic nanoparticles elutes from the column; and collecting an eluent comprising the selected portion of the plurality of inorganic nanoparticles.

Statement 8. A method of Statement 6, comprising depositing the selected portion of the plurality of inorganic nanoparticles in an HPLC column comprising an input in fluid communication with a stationary phase in fluid communication with an output in fluid communication with a detector; passing a mobile phase through the HPLC column, such that the selected portion of the plurality of inorganic nanoparticles elutes from the column and enters the detector, such that the detector generates a signal, wherein the signal indicates the location of the one or more dye on and/or in the individual inorganic nanoparticles of the selected portion of the plurality of the inorganic nanoparticles; analyzing the signal to determine the location of the one or more dye on and/or in the individual inorganic nanoparticles of the selected portion of the plurality of inorganic nanoparticles; and optionally, collecting one or more fraction of the eluent.

Statement 9. A method of any one of the preceding Statements, where the reaction mixture further comprises alumina or aluminosilicate core monomer and the pH of the reaction mixture is adjusted to a pH of 1 to 2 prior to addition of the alumina or aluminosilicate core forming monomer and, optionally, PEG is added to the reaction mixture prior to adjusting the pH to a pH of 7 to 9, and the core is an aluminosilicate core.

Statement 10. A method of any one of the preceding Statements, where the dye precursor is a positively charged dye precursor, a negatively charged dye precursor, or a net neutral dye precursor.

Statement 11. A method of Statement 10, where the positively charged dye precursor is formed from a positively charged dye chosen from Cy5.5, Cy5, Cy3, ATTO647N, methylene blue, ATTO663, ATTO620, ATTO665, ATTO465, ATTO495, ATTO520, ATTORho6G, ATTORho3B, ATTORho11, ATTORho12, ATTOThio12, ATTO580Q, ATTORho101, ATTORho13, ATTO610, ATTO612Q, ATTO647N, ATTORho14, ATTOOxa12, ATTO725, ATTO740, ATTOMB2, and the like, and combinations thereof.

Statement 12. The method of claim 10, wherein the negatively charged dye precursor is formed from a negatively charged dye chosen from sulfo-Cy5.5, sulfo-Cy5, sulfo-Cy3, Alexa Fluor 532, Alexa Fluor 430, ATTO430LS, ATTO488, ATTO490LS, ATTO532, ATTO594, and the like, and combinations thereof.

Statement 13. A method of Statement 10, wherein the net neutral dyes precursor is formed from a net neutral dye chosen from tetramethylrhodamine (TMR), ATTO390, ATTO425, ATTO565, ATTO590, ATTO647, ATTO650, ATTO655, ATTO680, ATTO700, and the like, and combinations thereof.

Statement 14. A composition comprising a plurality of inorganic nanoparticles, wherein the individual inorganic nanoparticles of the plurality of inorganic nanoparticles comprise 1-7 dye group(s), wherein: i) none of the dye groups are disposed or partially disposed on the surface of the inorganic nanoparticles; or ii) the majority (e.g., greater than 50%) of the inorganic nanoparticles have at least one (e.g., one, two, three, four, five, six, seven, or a combination thereof) dye group(s) disposed or partially disposed on the surface of the inorganic nanoparticles; or iii) all of the dye groups are disposed or partially disposed on the surface of the inorganic nanoparticles, where the dye group is positively charged, negatively charged, or has a net neutral charge.

Statement 15. A composition of Statement 14, where the plurality of inorganic nanoparticles consists essentially of individual inorganic nanoparticles having 0, 1, 2, 3, 4, 5, 6, or 7 dye group(s) encapsulated (e.g., fully encapsulated) in the inorganic nanoparticles.

Statement 16. A composition of Statement 14, where the one or more dye group is positively charged or has net neutral charge and the plurality of inorganic nanoparticles do not exhibit size-dependent surface inhomogeneity.

Statement 17. A composition of Statement 16, where size-dependent surface inhomogeneity is determined by HPLC.

The following example is presented to illustrate the present disclosure. It is not intended to be limiting in any matter.

### Example

The following in an example of synthesis and characterization of nanoparticles of the present disclosure.

Dyes exposed on the particle surface have a greater propensity for chemical degradation or potential hydrolysis and may therefore have significant effects on the behavior of these types of nanoparticles in complex biological environments such as the human body. Having identified a method to quantitatively assess these surface chemical heterogeneities as well as their main driver, i.e. dye charge, now allows us to perform systematic studies in order to provide insights into the factors in the interactions between ˜2 nm sized silica clusters originally formed in the sol-gel silica synthesis and the fluorescent dyes that govern optimal dye encapsulation.

Presented herein are new insights into the physical parameters that govern full covalent fluorescent dye encapsulation within the silica core of poly(ethylene glycol) coated core-shell silica nanoparticles referred to as Cornell Prime Dots (C′ dots). A combination of high-performance liquid chromatography (HPLC), gel permeation chromatography (GPC), and fluorescence correlation spectroscopy (FCS) was used to monitor the result of ammonia concentration in the synthesis of C′ dots from negatively and positively charged versions of near infrared dyes Cy5 and Cy5.5. HPLC in particular allows the distinction between cases of full versus partial dye encapsulation in the silica particle core leading to surface chemical heterogeneities in the form of hydrophobic surface patches, which in turn modulate biological response in ferroptotic cell death experiments. These results demonstrate that there is a complex interplay between dye-dye and dye-silica cluster interactions originally formed in the sol-gel synthesis governing optimal dye encapsulation. It is expected that the reduced surface chemical heterogeneities will make the resulting nanoparticles attractive for a number of applications in biology and medicine.

Described herein is negatively and positively charged variants of the NIR dyes Cy5 and Cy5.5 relevant for applications in nanomedicine. Combining HPLC with gel permeation chromatography (GPC) and FCS, synthesis conditions under which full dye encapsulation into the silica core is achieved for both dyes, leading to minimal heterogeneity in particle surface chemical properties obtained from individual synthesis batches. In particular, it was shown that for different dyes successful encapsulation requires careful tuning of the starting concentration of ammonium hydroxide in aqueous solution. This directly effects the rate of hydrolysis and condensation of the silica precursors, and the surface charge of the resulting silica clusters formed initially in the sol-gel process, which in turn governs the electrostatics of both cluster-dye and cluster-cluster interactions critical for successful particle formation. Finally, it is demonstrated how particle heterogeneities based on different degrees of dye encapsulation modulate biological response, using recently discovered C dot induced ferroptosis, an iron dependent cell death program, as a test bed.

Methods.

Chemicals and Reagents. UHPLC grade acetonitrile was purchased from BDH. Superdex 200 resin was purchased from GE Healthcare Life Sciences. Vivaspin 30k MWCO spin filters were purchased from GE Healthcare Life Sciences. 5 M NaCl in water solution was purchased from Santa Cruz Biotechnology. Dimethyl sulfoxide (DMSO), tetramethyl orthosilicate (TMOS), (3-mercaptopropyl)trimethoxysilane (MPTMS), Iron(III) nitrate, and 2.0 M ammonia in ethanol were all purchased from Sigma-Aldrich. Methoxy-PEG(6-9)-silane (˜500 g/mol) was purchased from Gelest. Sulfo-Cy5-maleimide and sulfo-Cy5.5-maleimide were purchased from GE. Cy5-maleimide and Cy5.5-maleimide were purchased from Lumiprobe. DI water was generated using a Millipore IQ7000 system (18.2 MΩ·cm). Xbridge Protein BEH C4 Column (300 Å, 3.5 μm, 4.6 mm×150 mm, 10K-500K) was purchased from Waters Technologies Corporation. MDA-MB-468 cells were obtained from the ATCC, and were used within 3 months of thawing. RPMI-1640, fetal bovine serum (FBS), and dialyzed FBC were from Gibco. Amino-acid-free RPMI-1640 was from United States Biological. GlutaMax, Pen/Strep, and PrestoBlue reagent were from Invitrogen. All chemicals were used as received without further purification.

Particle Synthesis and Purification. C′ dots were synthesized as previously described. Briefly, for a 10 mL batch, 0.367 μmol mono functional maleimido derivatized dye was dissolved in DMSO overnight in a glovebox. A 23-fold excess of mercaptopropyl-trimethoxysilane (MPTMS) was added to the dissolved dye and allowed to react overnight in the glove box. The next day a flask containing deionized water pH adjusted using between 0.5 mL and 2.5 mL of 0.02 M ammonium hydroxide (ammonium hydroxide was prepared by mixing 100 μL of 2.0 M ammonia in ethanol solution into 10 mL of deionized water) was prepared and stirred vigorously. For sulfonated dyed C′ dot syntheses, 1 mL of 0.02 M ammonium hydroxide was added into 9 mL deionized water. 68 μL tetramethylorthosilicate (TMOS) and the prepared dye-silane conjugate were added to the flask drop-wise and allowed to react overnight. The following day, 100 μL of mPEG(6-9)-silane was added to the flask drop-wise and allowed to react overnight. The following day stirring of the solution was stopped and the flask heated to 80° C. for 24 hours. Following this heating step, the particles were extensively dialyzed using 10K MWCO cellulose dialysis tubing, followed by syringe filtration with a 200 nm membrane, spin filtering with a 30K MWCO PES membrane spin filter, and finally GPC purification through Superdex 200 resin on a Bio-Rad FPLC. The particles were then characterized using fluorescence correlation spectroscopy (FCS) on a home-built setup and UV/Vis spectroscopy on a Cary 5000 spectrometer.

High Performance Liquid Chromatography (HPLC). All injections were performed at a standardized injection volume of 8 μL and concentration of 30 μM. Concentrations for injected samples were determined prior to analysis by FCS. The columns used were 150 mm Waters Xbridge BEH C4 Protein separation columns with 300 Å pore size and 3.5 μm particle size. The separation method used was as follows: The sample was first injected onto the column in a flow of 90:10 water:acetonitrile at a flow rate of 0.75 mL/min, and this isocratic segment of the method was maintained for 20 minutes. The mobile phase composition was then changed to 45:55 water:acetonitrile in a step-like fashion and the baseline was allowed to equilibrate for 5 minutes. Finally, a composition gradient of 45:55 to 5:95 water:acetonitrile was run over the course of 20 minutes. During this time, the analyte eluted from the column.

Gel Permeation Chromatography (GPC). Preparative scale gel permeation chromatography was carried out on a Bio-Rad FPLC equipped with a UV detector set to 275 nm. Particles were purified under isocratic conditions using 0.9 wt. % NaCl in deionized water. The eluent was prepared directly before nanoparticle purification by diluting 0.2 μm membrane filtered 5 M NaCl in water (Santa Cruz Biotechnology) with deionized water (18.2 M2 acquired from Millipore IQ7000). The column used was a standard glass Bio-Rad column with dimensions of 20 mm×300 mm and was hand-packed with Superdex 200. The column was operated at 2.0 mL/min and was allowed to equilibrate with the mobile phase for at least 30 minutes before sample purification. All samples were concentrated in GE Life Sciences 30 kDa MWCO VivaSpin filters prior to injection. The total injection volume was less than 1 mL per run. Particles eluted around the 15-minute mark and the total run lasted 30 minutes.

Fluorescence Correlation Spectroscopy (FCS). FCS measurements were carried out on a home built set up using a 635 nm solid state laser and a continuous wave laser. This is the standard laser setup for fluorescent dyes with absorbance maxima around 650 nm. The continuous wave laser was focused onto the image plane of a water immersion microscope objective (Zeiss Plan-Neofluar 63×NA 1.2). The emitted fluorescence was Stokes-shifted and therefore after it passed back through the same objective it could successfully pass through a dichroic mirror, after which it was spatially filtered by a 50 m pinhole and then finally through a spectrally filtered long pass filter (ET665lp, Chroma) before being detected by an avalanche photodiode detector (SPCM-AQR-14, PerkinElmer). The signal was autocorrelated by a digital correlator (Flex03LQ, Correlator.com) with a lag time resolution of 15 ns. Autocorrelation curves were fitted with equation (1) that accounts for fast photophysical processes and translational diffusion.

$$G ( τ ) = 1 + \frac{1}{{N}_{m}} ( \frac{1}{1 + τ / {τ}_{D}} ) {( \frac{1}{1 + τ / ( {τ}_{D} {κ}^{2} )} )}^{1 / 2} \frac{1}{( 1 - P )} ( 1 - P + P exp ( τ / {τ}_{P} ) ) \qquad ( 1 )$$

Where Nₘ is the number of fluorescent particles diffusing through the focal volume at any given time, τ_{D} is the average translational diffusion time of the fluorescent material diffusing through the focal volume, τ_{P} is the characteristic relaxation time for fast photophysical processes, κ is the focal volume structure factor calculated from the radial and axial radii (κ=ω_{z}/ω_{xy}), and P is the fraction of fluorescent particles undergoing a fast photophysical process during the experiment. All autocorrelation curves were normalized according to equation (2):

G(τ)=(G(τ)−1)Nₘ (2)

UV/Vis Spectroscopy. Absorbance spectra of C′ dot samples were measured in DI water on a Varian Cary 5000 spectrophotometer in a 3 mL quartz cuvette with a 10 mm light path (HellmaAnalytics) from 200 nm to 800 nm in 1 nm increments. All spectra were baseline corrected using a cuvette with DI water as reference cell. Maximum absorption at the dye absorption wavelength was kept between 0.01 and 0.06.

Cell Work. MDA-MB-468 cells were maintained at 37 degrees Celcius, 5% C02, in complete medium (RPMI-1640 supplemented with 10% FBS). Cells were plated in 96 well plates at a concentration of 2×10⁴ cells per well and allowed to settle overnight. The medium was then removed, and replaced with amino-acid-free RPMI-1640 supplemented with 10% dialyzed FBS, 1×GlutaMax, and 1×Pen/Strep, along with the indicated amount of iron(III) nitrate or C′ dots. In experiments with C′ dots, all conditions also included 1 μM iron(III) nitrate. The cells were incubated for 6 days. The medium was then replaced with complete medium, and cell viability was assessed with PrestoBlue reagent per the manufacturer's instructions. Data was read out in absorbance mode on a Tecan Safire instrument.

Results and Discussion.

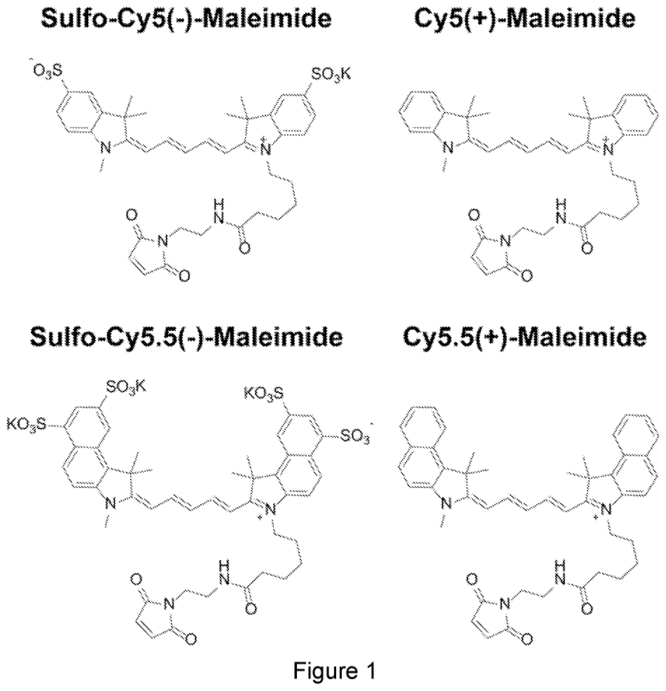

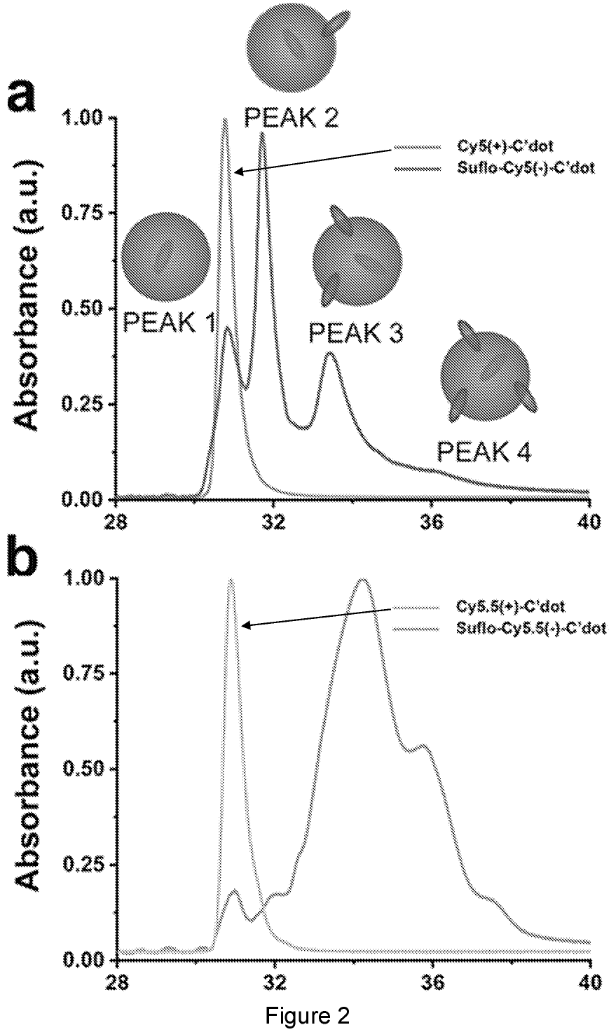

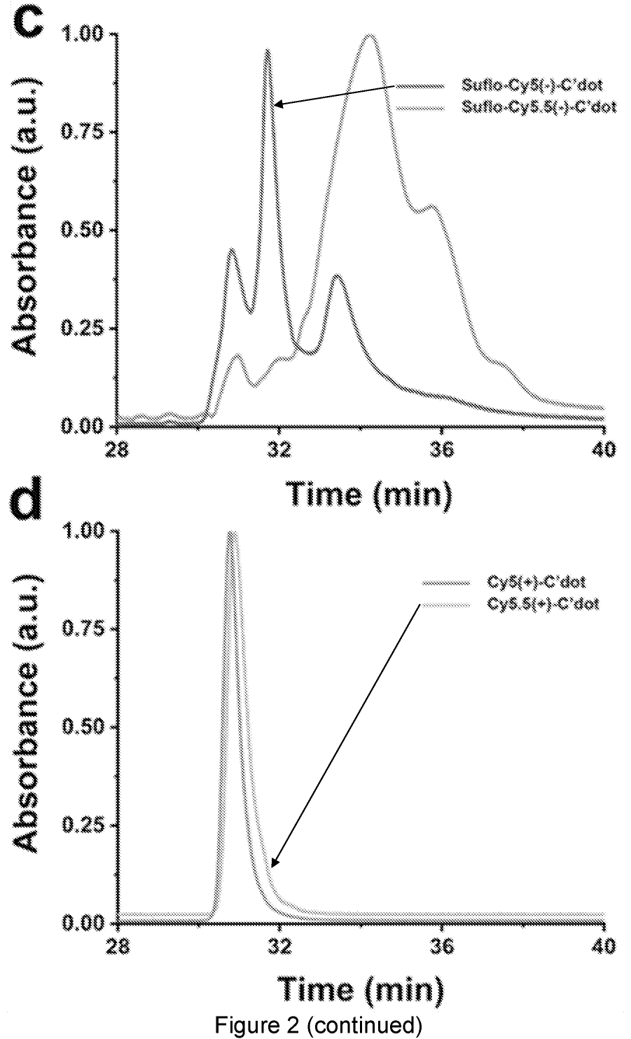

Positively Charged Dye Chemistry Greatly Improves Particle Surface Chemical Homogeneity. FIG. 1 shows sulfonated Cy5 that has a net charge of −1 (top row, left). As discussed in the introduction and shown in FIG. 2a, because of repulsive Coulomb interactions with negatively charged silica at slightly basic synthesis conditions, this net negative charge causes a significant amount of nanoparticles surface chemical heterogeneity as manifested in the occurrence of three prominent and one weak peak in HPLC chromatograms. As indicated in the insets of FIG. 2a, these peaks correspond to particles with 0, 1, 2, or 3 dyes covalently attached to the silica surface (FIG. 2a). By switching to the unsulfonated derivative of Cy5 (FIG. 1, top row, right), dye charge in solution changes from −1 to +1, which as shown in FIG. 2a promoted full encapsulation as the result of what are now attractive dye-silica interactions. As the HPLC chromatogram shows, the positively dye encapsulating particles all eluted at the same time giving rise to a single peak reflecting high particle surface homogeneity.

While the surface chemical heterogeneity of C′ dots caused by encapsulation of a dye with net −1 charge has been investigated via HPLC and is now understood, the effects on particle surface heterogeneity of dyes with even higher net negative charge is still an outstanding question. This is particularly relevant for near infrared (NIR) dyes such as Cy5.5 (abs./em.: 675/695 nm), which are larger than Cy5 (abs./em.: 650/670 nm) in order to emit further out in the NIR and therefore are significantly more hydrophobic. The commercially available sulfonated analogue of Cy5.5 carries four sulfonate groups and has a net charge of −3 (FIG. 1, bottom row, left) in order to provide good solubility in aqueous solutions despite the large hydrophobic molecular framework. This poses a significant challenge to the encapsulation into ultrasmall fluorescent core-shell silica nanoparticles due to strong repulsive interactions between the highly negatively charged primary silica clusters initially formed in the sol-gel synthesis of C′ dots and the negatively charged dyes. The negative charge of the primary clusters in the pH range where the C′ dot synthesis is typically performed is attenuated as they aggregate and as more silicic acid is produced via hydrolysis of TMOS. According to previous molecular dynamics simulations, it is this decrease in cluster surface charge as the synthesis proceeds which eventually allows the negatively charged dyes to condense onto the surface of the forming C′ dots. The HPLC chromatogram of C′ dots synthesized with sulfo-Cy5.5 is shown in FIG. 2b. Compared to that of the sulfo-Cy5 derived particles (FIG. 2C), it displays dramatically increased heterogeneity, as manifested in at least five peaks that extend to much longer elution times indicating a significantly more hydrophobic particle. It is clear from this data, that the majority of the dyes in the Cy5.5 based C′ dot synthesis ended up on the particle surface.

Primary Cluster Charge is a Critical Parameter to Control. While switching from a negative dye to a positive dye analogue generally affords significant enhancement in nanoparticle homogeneity, synthesis parameters must be tuned as a function of individual dye chemistries to ensure optimal nanoparticle surface chemical properties. Switching to the positively charged, unsulfonated analogues of dyes like Cy5 and Cy5.5 allows for the dyes to act as nucleation sites for the nanoparticles formed from negatively charged primary silica clusters, which is responsible for the full encapsulation into silica as the C′ dots grow. In turn, for fully optimized synthesis conditions (vide infra) this leads to a high degree of surface chemical homogeneity as evidenced by single peaks in HPLC chromatograms as shown in FIGS. 2a and b for Cy5 and Cy5.5, respectively. However, this switch of dye chemistry is not without challenges, as the positively charged NIR dyes do not have the benefit of increased water solubility of the sulfonated dye analogues, and as a result are prone to aggregation in water. In order to optimize NIR C′ dot synthesis conditions we turned to a combination of gel permeation chromatography (GPC), high performance liquid chromatography (HPLC), and fluorescence correlation spectroscopy (FCS). The optimization of ultrasmall fluorescent core-shell silica nanoparticles with covalently encapsulated positive NIR dyes is more nuanced than the straightforward transfer from negatively charged dyes to positively charged dyes, but equally important in order to ensure a homogeneous PEGylated nanoparticle surface. Three main interactions must be taken into account for this process: dye-dye interactions, dye-primary cluster interactions, and cluster-cluster interactions. Dye-dye interactions are more important with unsulfonated, positively charged dyes because the dyes are more hydrophobic and prone to aggregation. The concentration of dye must be carefully controlled to not cause too much dye aggregation and precipitation, which will prevent efficient dye encapsulation, while maintaining a high enough free dye concentration to provide a sufficiently high synthesis yield.

The positively charged Cy5 and Cy5.5 dyes likely act as nucleation sites for the negatively charged ˜2 nm primary silica clusters that form when the silica precursor, TMOS, is added into the aqueous reaction solution. This promotes the growth of nanoparticles and the subsequent dye encapsulation, vide supra. As primary clusters aggregate around a positively charged dye, the positive charge becomes electrostatically shielded and the growing nanoparticle becomes more repulsive towards additional cluster association as the particles reach electrostatic stabilization via growth. If the ammonia concentration in the synthesis solution is sufficiently high, these repulsive cluster-cluster interactions between highly negatively charged clusters may stop further cluster addition before the dye is fully encapsulated within silica primary clusters. This would lead to overall smaller nanoparticles and larger numbers of hydrophobic dye patches on the surface.

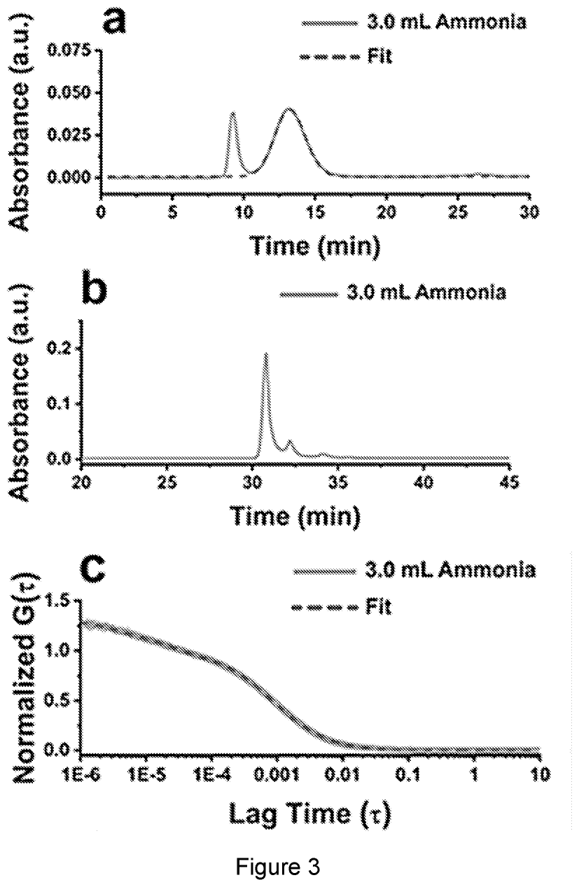

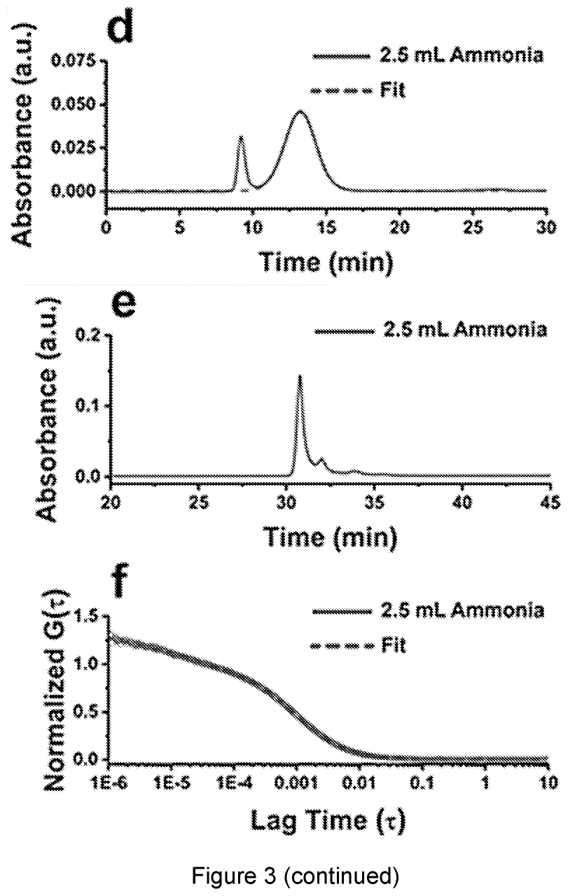

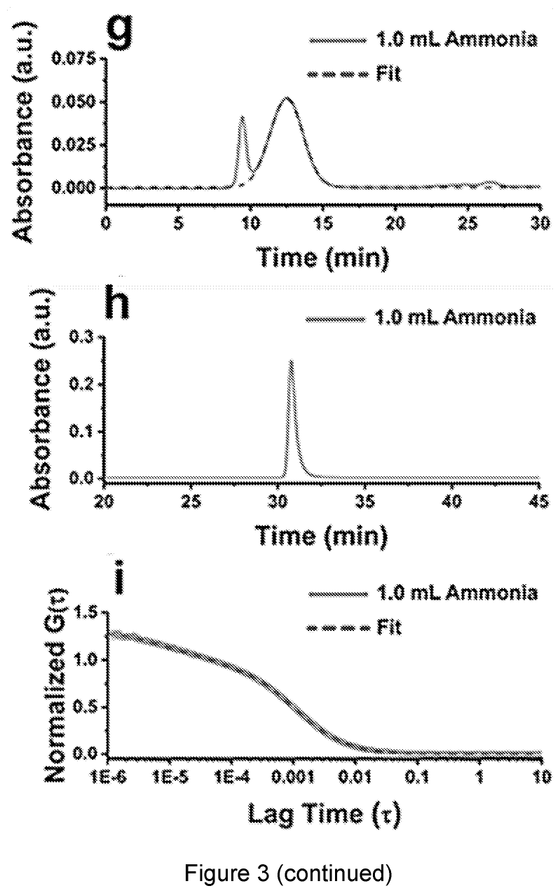

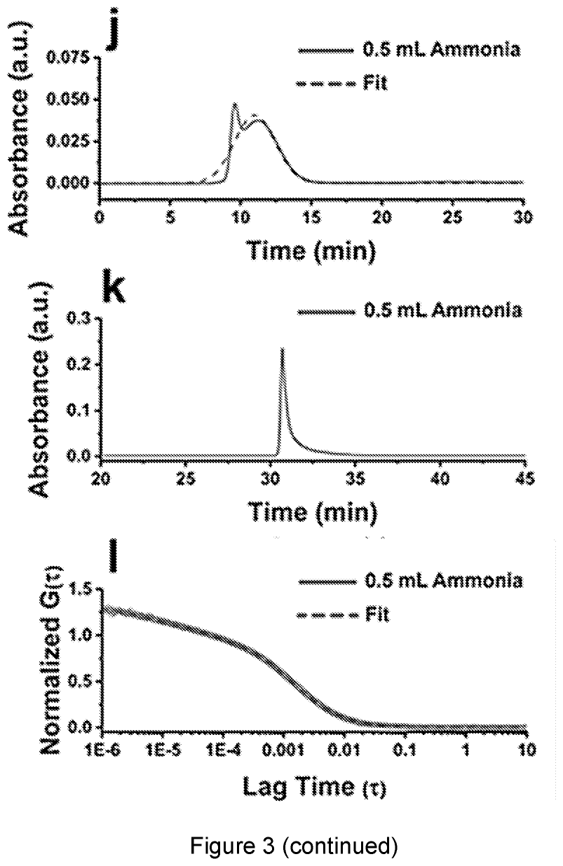

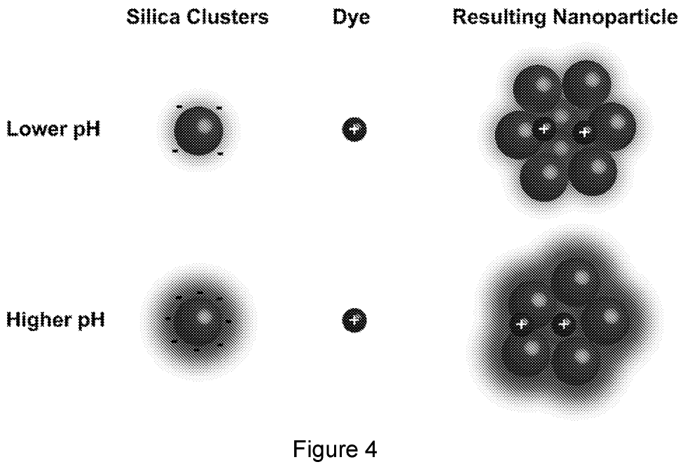

Surface Chemistry Optimization for covalent encapsulation of NIR Dye Cy5(+). This is what is observed in C′ dot synthesis experiments with positively charged Cy5 [Cy5(+)]: For increasing ammonia concentration the main nanoparticle peak in GPC moves to the right (FIG. 3a,d,g,j) indicating smaller particles as corroborated by FCS (FIG. 3c,f,i,l; Table 1); the corresponding HPLC traces (FIG. 3b,e,h,k) show enhanced heterogeneity via additional peaks at higher elution times. To prevent the formation of nanoparticles with only partially encapsulated dyes, the surface charge of the primary silica clusters that form the core of the C′ dots must be carefully controlled by modulating the concentration of ammonium hydroxide in solution. Ammonium hydroxide is a catalyst for the basic hydrolysis and condensation of silica in the C′ dot synthesis. It not only controls the rate of hydrolysis and condensation, but also the surface charge of the primary silica clusters formed at the beginning of the sol-gel synthesis. Meanwhile, if the ammonium hydroxide concentration is too low, the primary silica clusters will be significantly more prone to aggregation. While these nanoparticles will totally encapsulate the dye, the size increases significantly due to uncontrolled aggregation of primary silica clusters. This leads to the primary nanoparticle peak and the nanoparticle aggregate peak in GPC becoming inseparable as shown in FIG. 3j. As previously demonstrated, even with extremely careful purification via GPC fractionation, the nanoparticle peak will always contain some aggregates that cannot be separated out. As demonstrated in FIG. 3g-i, when working with positive Cy5-silane, the optimal starting primary cluster surface charge was reached for 10 mL batches (see Methods) at a concentration of 1.0 mL (of 0.02 ammonia solution). Higher starting ammonia concentrations caused the dye to not be fully encapsulated, while lower ammonia concentration caused the clusters to aggregate too much due to very low surface charge. FIG. 4 illustrates this principle of balancing primary silica cluster charge and dye charge and resulting encapsulation effects.

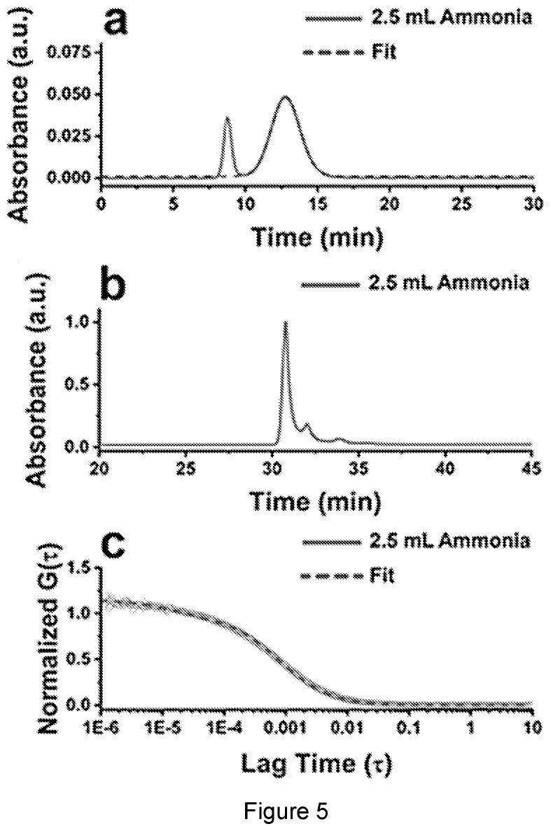

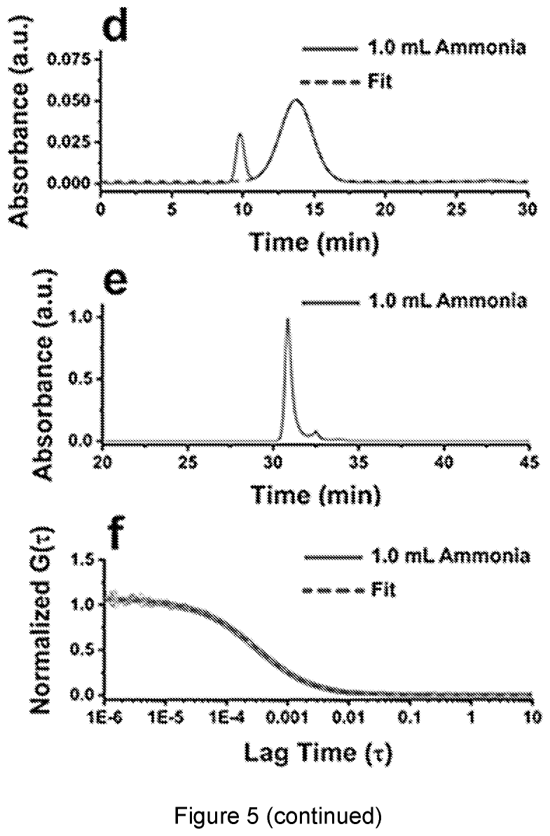

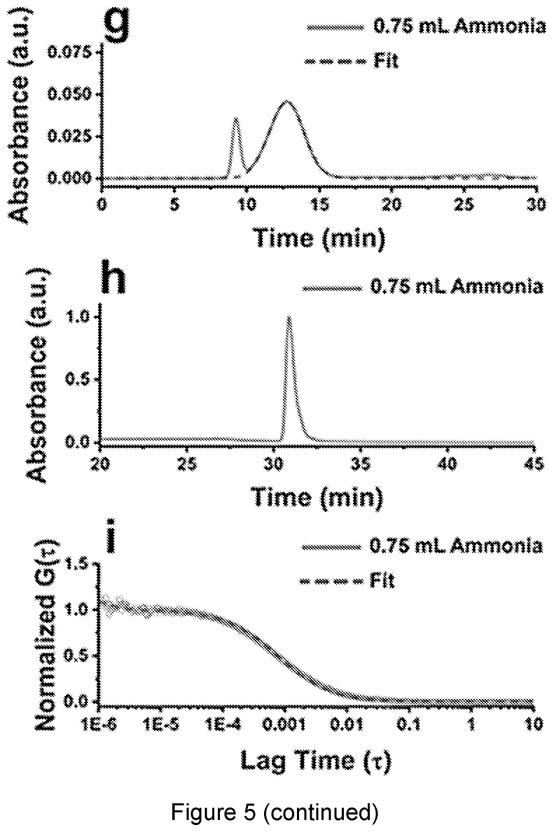

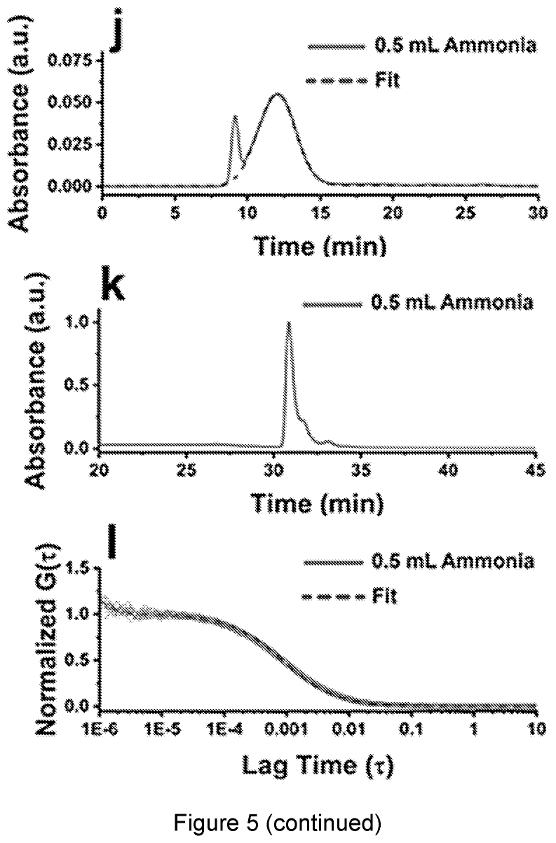

Surface Chemistry Optimization for covalent encapsulation of NIR Dye Cy5.5(+). Next, this principle was applied to optimize the synthesis of NIR C′ dots from positively charged Cy5.5 [Cy5.5(+)], a second clinically relevant variant of the C′ dots. Evaluation of Cy5.5 C′ dot syntheses along the lines of what was discussed for Cy5(+) based C′ dots revealed that as a result of the different dye chemistry, the optimal synthesis conditions for Cy5.5(+) are different. Positively charged Cy5.5 is significantly larger and more hydrophobic than Cy5(+), and as a result, adjustments had to be made to the synthetic protocol. Comparing GPC, HPLC, and FCS results for different ammonia concentrations in the synthesis (FIG. 5), for a 10 mL batch the optimal ammonia concentration for Cy5.5 C′ dots now was around 0.75 mL (of 0.02 M ammonia solution) rather than around 1 mL for Cy5(+), see FIG. 5g-i.

The optimization of Cy5.5 C′ dots highlights that for a given dye in order to obtain full dye encapsulation there is an optimal surface charge for the primary silica clusters. Simply decreasing the ammonia concentration at the beginning of the reaction is not a straightforward solution. As the pH at the beginning of the synthesis decreases as a result of TMOS hydrolysis and silicic acid formation (vide supra), the solubility of positively charged dyes increases, which leads to a higher propensity for dyes to condense onto the surface of the growing nanoparticles. As demonstrated with the help of GPC, HPLC, and FCS results in FIG. 5j-1, when using 0.5 mL of ammonia solution in the reaction, the particle size further increases relative to the results for 1 mL. But this time the surface heterogeneity of the particles increases too (see FIG. 5k). In contrast, Cy5(+)-C′ dots had homogeneous surface chemistry even below the optimal ammonium hydroxide concentration (compare FIG. 3k with 5k).

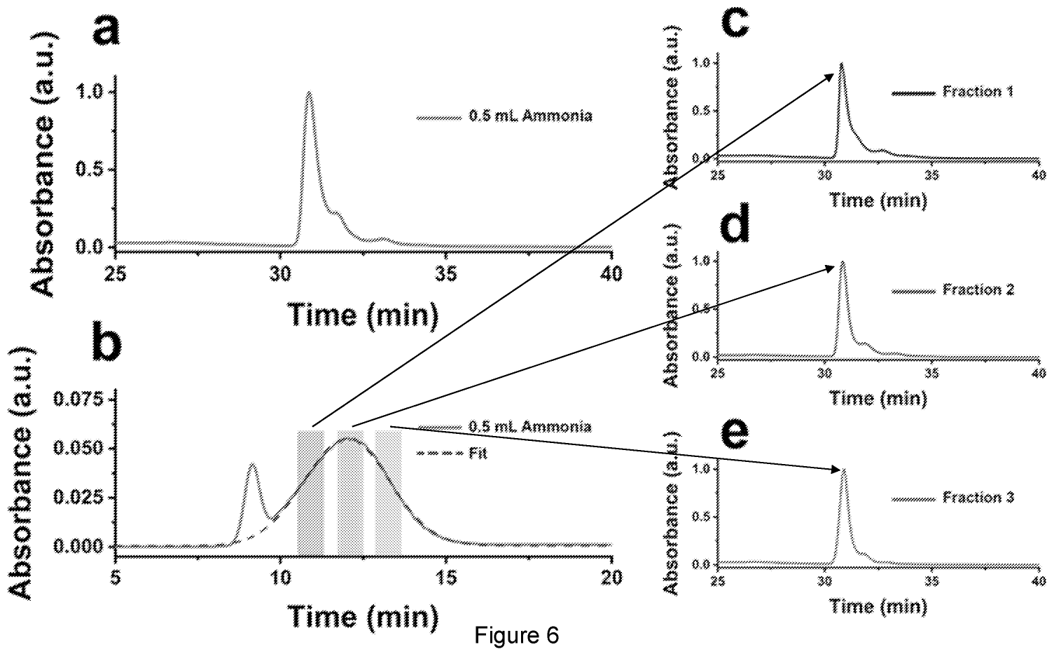

GPC-HPLC Elucidates Additional Heterogeneity for Low Deprotonation Conditions. The case of Cy5.5(+) highlighted the importance of correctly optimizing the ammonium hydroxide concentration of the synthesis solution for each individual dye. In order to further understand the origin of the heterogeneity that occurred in the regime of low primary silica cluster surface charge, we GPC-fractionated the sample of PEG-Cy5.5(+)-C′ dots that showed reoccurring surface heterogeneity at 0.5 mL of 0.02 M ammonium hydroxide in the synthesis (FIG. 6a,b), which is below the optimal ammonia concentration for Cy5.5(+) dye (0.75 mL). Three GPC fractions were then analyzed with HPLC (FIG. 6c-e) showing surface heterogeneity is increased for earlier time fractions that reflect larger particle size, suggesting extra Cy5.5 dye condensing on the nanoparticle surface, and creating extra hydrophobic patches before the nanoparticle PEGylation step. Interestingly, the GPC trace reflecting the particle size distribution could still be well fitted with a single Gaussian function (FIG. 6b), whereas this was not the case for Cy5(+) synthesis where the GPC peak was skewed below 1 mL ammonium hydroxide condition (compare quality of fits in FIGS. 3j and 5j). This suggests that at 0.5 mL ammonia synthesis conditions, after dye-mediated initial dye-cluster conjugate formation the particle growth mechanism of Cy5(+)-C′ dots is primarily based on the continued addition of primary silica clusters to the growing nanoparticles, whereas in the Cy5.5(+)-dot synthesis continued addition of primary silica clusters is accompanied by extra dye condensation onto the nanoparticle surface. These results together demonstrate that simultaneous control of dye-dye and dye-silica interactions during C′ dot synthesis is highly non-trivial, and synthesis conditions must be carefully tuned for any new dye candidate to achieve optimal fluorescent core-shell silica nanoparticle formation with minimal surface chemical heterogeneity.

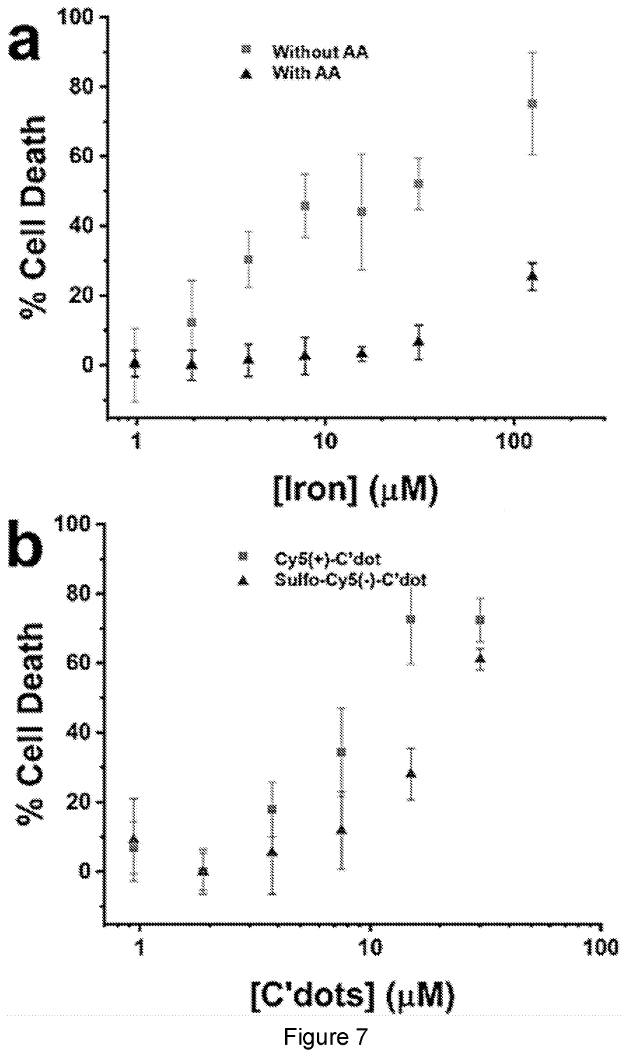

Different Degrees of Surface Chemical Heterogeneity Modulate Biological Response. It was determined whether nanoparticle heterogeneity in the form of hydrophobic surface patches from non-encapsulated NIR dyes had any effect on biological response. To that end, C′ dot induced iron-mediated cell death program ferroptosis observed under nutrient deprivation conditions of cancer cell populations, in which the core-shell silica nanoparticles, as a result of micropores of the silica core, chelate iron from solution and carry it into the cancer cells was chosen as a test bed. The sensitivity of MDA-MB-468 triple-negative breast cancer cells to treatment with iron(III) nitrate under amino-acid (AA) starved conditions was assessed. As shown in FIG. 7a, the cells were insensitive to 1 μM iron, but in contrast to full medium controls near-total cell death was elicited by 6 μM iron. To determine the degree to which C′ dots could modulate this biological response, we treated the cells with assorted concentrations of C′ dots, while in the presence of a non-toxic amount of iron (1 μM). As demonstrated in FIG. 7b, the particles prepared with positively charged Cy5 dye were able to elicit a cell death response much greater than 1 μM iron alone, in particular around and above 10 μM particle concentrations, while particles prepared with negatively charged sulfo-Cy5 dye were able to elicit only a relatively small cell death response. Thus, the positively charged Cy5 dye encapsulating particles seem to be superior at introducing iron into the cancer cells and inducing ferroptotic cell death likely due to the decreased hydrophobic surface patchiness of these particles and associated facilitated access to the micropores of the silica core.

Conclusion. Control parameters in the synthesis of fluorescent dye-encapsulating core-shell silica nanoparticles that determine surface chemical particle heterogeneities were elucidated. The present disclosure highlights that depending on the dye used, small variations in synthetic conditions, here the concentration of ammonia as a catalyst in the sol-gel reaction, can lead to significant changes in surface chemical properties in the form of hydrophobic surface patches resulting from dyes conjugated to the silica core surface rather than being fully encapsulated. The complex interplay of dye-dye and dye-silica cluster interactions is critical to understand in order to effectively control the surface chemistry of the final nanoparticles. Variations in particle surface chemical properties/heterogeneities in turn modulate biological response to the nanoparticles, as demonstrated by ferroptotic cell death experiments with C′ dots derived from either negatively and positively charged Cy5 dye. Without intended to be bound by any particular theory, it is considered that these synthetic insights into the nucleation and growth of fluorescent core-shell silica nanoparticles prepared in aqueous solutions will have implications for application of such nanoparticles in bioimaging and nanomedicine.

TABLE 1
Tabulated results of the FCS analysis
of the samples shown in FIGS. 3 and 4.

| | Volume of | | Brightness | Dyes Per |
|---|---|---|---|---|
| | Ammonia | | Per Particle | Particle |
| Dye | [mL] | Size [nm] | [kHz] | [#] |
| Cy5(+)- | 0.50 | 8.6 ± 0.3 | 2.42 × 10⁴ | 2.0 |
| Maleimide | 1.00 | 6.3 ± 0.2 | 2.27 × 10⁴ | 1.9 |
| | 2.50 | 5.5 ± 0.1 | 2.22 × 10⁴ | 1.6 |
| | 3.00 | 5.3 ± 0.0 | 2.14 × 10⁴ | 1.9 |
| Cy5.5(+)- | 0.50 | 6.1 ± 0.1 | 4.02 × 10³ | 2.4 |
| Maleimide | 0.75 | 5.5 ± 0.1 | 4.16 × 10³ | 2.5 |
| | 1.00 | 5.5 ± 0.1 | 4.16 × 10³ | 2.5 |
| | 2.50 | 5.0 ± 0.1 | 3.91 × 10³ | 1.8 |

Although the present disclosure has been described with respect to one or more particular embodiments and/or examples, it will be understood that other embodiments and/or examples of the present disclosure may be made without departing from the scope of the present disclosure.

1. A method for synthesizing an inorganic nanoparticle comprising one or more dyes and surface functionalized with polyethylene glycol (PEG) groups, comprising:
a) forming a reaction mixture at room temperature comprising water, TMOS, a base, and a dye precursor;
b) either i) holding the reaction mixture at a time (t¹) and temperature (T¹), whereby inorganic nanoparticles having an average size of 2 to 15 nm are formed, or ii) cooling the reaction mixture to room temperature, if necessary, and adding a shell forming monomer to the reaction mixture from a), whereby inorganic nanoparticles have a core size of 2 to 15 nm and/or an average size of 2 to 50 nm are formed;
c) adjusting, if necessary, the pH of the reaction mixture comprising the inorganic nanoparticles from b) i) or b) ii) to a pH of 6 to 10;
d) adding at room temperature to the reaction mixture comprising the inorganic nanoparticles from b) i) or b) ii) a PEG-silane conjugate and holding the resulting reaction mixture at a time (t²) and temperature (T²);
e) heating the mixture from d) at a time (t³) and temperature (T³), whereby the inorganic nanoparticles surface functionalized with PEG groups are formed; and
f) purifying the reaction mixture by liquid chromatography, wherein the mole ratio of the dye precursor, TMOS, the base, and the PEG-silane is 0.0090-0.032:11-46:0.5-1.5:5-20, and
the method yields the inorganic nanoparticle comprising one or more dyes and surface functionalized with polyethylene glycol (PEG) groups.

2. The method of claim 1, wherein the base is chosen from ammonium hydroxide, ammonia in ethanol, triethyl amine, sodium hydroxide, potassium hydroxide, and combinations thereof.

3. The method of claim 1, wherein the base has a concentration and the concentration is 0.001 mM to 60 mM.

4. The method of claim 1, wherein the purifying comprises isolating a selected portion of a plurality of inorganic nanoparticles from the reaction mixture.

5. The method of claim 1, further comprising analyzing the selected portion of the plurality of inorganic nanoparticles via gel permeation chromatography (GPC).

6. The method of claim 1, further comprising analyzing the selected portion of the plurality of inorganic nanoparticles via high performance liquid chromatography (HPLC).

7. The method of claim 1, wherein the purifying comprises:
depositing a plurality of inorganic nanoparticles in a chromatography column comprising an input in fluid communication with a stationary phase in fluid communication with an output in fluid communication with a detector;
passing a mobile phase through the chromatography column, such that the plurality of inorganic nanoparticles elutes from the column; and
collecting an eluent comprising the selected portion of the plurality of inorganic nanoparticles.

8. The method of claim 6, comprising:
depositing the selected portion of the plurality of inorganic nanoparticles in an HPLC column comprising an input in fluid communication with a stationary phase in fluid communication with an output in fluid communication with a detector;
passing a mobile phase through the HPLC column, such that the selected portion of the plurality of inorganic nanoparticles elutes from the column and enters the detector, such that the detector generates a signal, wherein the signal indicates the location of the one or more dye on and/or in the individual inorganic nanoparticles of the selected portion of the plurality of the inorganic nanoparticles;
analyzing the signal to determine the location of the one or more dye on and/or in the individual inorganic nanoparticles of the selected portion of the plurality of inorganic nanoparticles; and
optionally, collecting one or more fraction(s) of the eluent.

9. The method of claim 1, wherein the reaction mixture further comprises alumina or aluminosilicate core monomer and the pH of the reaction mixture is adjusted to a pH of 1 to 2 prior to addition of the alumina or aluminosilicate core forming monomer and, optionally, PEG is added to the reaction mixture prior to adjusting the pH to a pH of 7 to 9, and the core is an aluminosilicate core.

10. The method of claim 1, wherein the dye precursor is a positively charged dye precursor, a negatively charged dye precursor, or a net neutral dye precursor.

11. The method of claim 10, wherein the positively charged dye precursor is formed from a positively charged dye chosen from Cy5.5, Cy5, Cy3, ATTO647N, methylene blue, ATTO663, ATTO620, ATTO665, ATTO465, ATTO495, ATTO520, ATTORho6G, ATTORho3B, ATTORho11, ATTORho12, ATTOThio12, ATTO580Q, ATTORho101, ATTORho13, ATTO610, ATTO612Q, ATTO647N, ATTORho14, ATTOOxa12, ATTO725, ATTO740, ATTOMB2, and combinations thereof.

12. The method of claim 10, wherein the negatively charged dye precursor is formed from a negatively charged dye chosen from sulfo-Cy5.5, sulfo-Cy5, sulfo-Cy3, Alexa Fluor 532, Alexa Fluor 430, ATTO430LS, ATTO488, ATTO490LS, ATTO532, ATTO594, and combinations thereof.

13. The method of claim 10, wherein the net neutral dyes precursor is formed from a net neutral dye chosen from tetramethylrhodamine (TMR), ATTO390, ATTO425, ATTO565, ATTO590, ATTO647, ATTO650, ATTO655, ATTO680, ATTO700, and combinations thereof.

14. The method of claim 1, wherein the one or more dyes are fully encapsulated in the inorganic nanoparticle.

15. A composition comprising a plurality of inorganic nanoparticles, wherein the individual inorganic nanoparticles of the plurality of inorganic nanoparticles comprise 1-7 dye group(s), wherein:
i) none of the dye groups are disposed or partially disposed on the surface of the inorganic nanoparticles; or
ii) greater than 50% of the inorganic nanoparticles have at least one dye group(s) disposed or partially disposed on the surface of the inorganic nanoparticles; or
iii) all of the dye groups are disposed or partially disposed on the surface of the inorganic nanoparticles,
wherein the dye group is positively charged, negatively charged, or has a net neutral charge.

16. The composition of claim 15, wherein the plurality of inorganic nanoparticles consists essentially of individual inorganic nanoparticles having 0, 1, 2, 3, 4, 5, 6, or 7 dye group(s) fully encapsulated in the inorganic nanoparticles.

17. The composition of claim 15, wherein the one or more dye group is positively charged or has net neutral charge and the plurality of inorganic nanoparticles do not exhibit size-dependent surface inhomogeneity.

18. The composition of claim 17, wherein size-dependent surface inhomogeneity is determined by HPLC.
